(19) United States
(12) Patent Application Publication
(10) Pub. No.: US 2026/0216079 A1
(43) Pub. Date: Jul. 30, 2026

(54) POLYMERIC MICROPARTICLES ENCAPSULATED WITH ACTIVE PHARMACEUTICAL INGREDIENTS AND RELATED METHODS OF USE AND MANUFACTURE

(71) Applicant: The General Hospital Corporation, Boston, MA (US)

(72) Inventors: Orhun Muratoglu, Boston, MA (US); Dinesh Dhamecha, Boston, MA (US); Mehmet D. Asik, Boston, MA (US); Ebru Oral Bhatia, Boston, MA (US)

(21) Appl. No.: 19/146,806

(22) PCT Filed: Jan. 9, 2024
(86) PCT No.: PCT/US2024/010874

§ 371 Date: Jul. 9, 2025

Related U.S. Application Data
(60) Provisional application No. 63/479,040, filed on Jan. 9, 2023.

(51) Int. Cl. A61K 9/16; A61K 9/00; A61K 31/496; A61K 31/7036; A61K 38/14

(52) U.S. Cl. CPC A61K 9/1647; A61K 9/0019; A61K 9/1694; A61K 31/496; A61K 31/7036; A61K 38/14

(57) ABSTRACT

Methods of delivering pharmaceutically active ingredients to patients and related methods of treatment are provided involving administering form of an active pharmaceutical ingredient that is inside polymeric microparticles. Related methods of manufacturing the microparticles with microfluidic devices are disclosed using double emulsion and/or single emulsion techniques. Hydrophilic and hydrophobic active ingredients may be provided in the polymeric microparticles and characteristics such as the in vivo release profile of the active ingredient may be tuned as desired.

### CROSS-REFERENCE TO RELATED APPLICATIONS

The present patent application claims priority to and the benefit of U.S. provisional patent application Ser. No. 63/479,040, filed on Jan. 9, 2023, the entire contents of which are herein incorporated by reference.

### FIELD OF THE INVENTION

This disclosure relates to active pharmaceutical ingredients (APIs) such as antibiotics, non-steroid anti-inflammatory drugs (NSAIDs), analgesics, and other drugs encapsulated in microparticles for treatment, inhibition or prevention of certain conditions or indications along with related methods of use and manufacture, including, for instance, use to prevent, treat, or inhibit chronic pain, among other conditions.

### BACKGROUND

Infectious diseases are disorders caused by organisms such as bacteria, viruses, fungi, or parasites. Often infections are associated with many types of surgeries resulting in surgical site infections (SSIs) and/or in some cases of peri-implant infections (PPIs) if an implant such as trauma plates, total joint implants, implantable pacemakers, catheters, etc. are used in the surgical procedure. Antibiotics are widely used to prophylactically prevent or treat these infections. Antibiotics are commonly applied orally, intravenously, intramuscularly, and/or intraarticularly. Typically, these applications do not use long-term control release (or elution) of antibiotics, compromising the potential prophylaxis and treatment that can be achieved with these therapeutic agents. The present disclosure addresses these needs with active pharmaceutical ingredients such as antibiotics, analgesics, and/or antimicrobials encapsulated in polymeric microparticles that can be delivered to the site of interest or need to ensure sustained long-term local delivery of these APIs.

Treatment of SSIs or PPIs could be improved with controlled local release of antibiotics. The postoperative incidence of SSI is about 2% to 4%. SSI can be either superficial, deep, or inside the organs. The most common germs causing SSIs include the bacteria Staphylococcus, Streptococcus, and Pseudomonas. The incidence of PPIs is much higher than SSIs and could range anywhere from 1% to 30%.

Infections that occur in total joint arthroplasty (TJA) patients, also known as periprosthetic joint infections (PJI), are characterized by pain, swelling, and tenderness of the affected area. PJI occurs in 1-2% of TJAs performed at tertiary health care facilities and at a higher rate at community hospitals. PJI has a remarkably high mortality rate due to the need for multiple revision surgeries. They have a recurrence rate of about 16% and can result in multiple resection arthroplasty, arthrodesis, and amputation. It is one of the leading causes of arthroplasty failure.

According to the current standard of care, PJI is managed with a two surgical procedures. The first surgery involves the removal of all infected tissue, hardware, and foreign material followed by irrigation and debridement of infected tissue. One or more antibiotic-impregnated spacer implants are inserted during the first surgery to treat the infection locally, in addition to intravenous and systemic administration of antibiotics. The second surgery involves the removal of the spacer implant(s) and implantation of a new prosthesis after approximately 4 months of continued treatment and surgical recovery. Patients are also put on oral and/or IV antibiotics (e.g. vancomycin, gentamicin, tobramycin, ceftriaxone, cefazolin, daptomycin, methicillin, cefazolin, oxacillin, nafcillin, and their salts) as part of the two-stage surgical treatment; however, the bioavailability of oral/IV antibiotics at the infection site can be limited. Increased dosing of oral/IV antibiotics leads to high serum levels of antibiotics and can be associated with serious systemic toxicity (e.g., ototoxicity or nephrotoxicity for antibiotics commonly used to treat PJI).

Another incidence where controlled release of APIs, such as analgesics and other therapeutic agents, from the microparticles of the current invention could be beneficial is in pain management. Effective pain management helps reduce suffering, promote healing and rehabilitation, and minimize complications in surgical patients. Patients are typically put on oral and/or IV drugs (fentanyl, hydromorphone, morphine, oxycodone, oxymorphone, tramadol, lidocaine, bupivacaine, ibuprofen, naproxen sodium, celecoxib, ketorolac, acetaminophen, ketamine, etc.) for pain management. The bioavailability of these oral/IV drugs at the surgical site can be limited and increasing oral or IV dose can lead to systemic toxicity, addiction, even mortality.

Concerns associated with frequent oral administration and systemic injections of therapeutic agents can be addressed with localized sustained delivery of these agents, such as antibiotics, non-steroid anti-inflammatory drugs, analgesics, and other drugs.

One technique of local controlled delivery of drugs is through encapsulation of these drugs in poly lactic-glycolic acid microparticles, which can be injected into the site of interest. It is known to manufacture PLGA microparticles to encapsulate drugs using single emulsion and double emulsion techniques. The first and most well-known method is the homogenization technique. Our research and others' published results have shown that the homogenization method leads to poor dispersity of particles, low loading capacity, and inefficient scalability.

There are examples of FDA-approved injectables for the controlled local release of various drug formulations. Nanoparticles can help release drugs in 1-2 days. For example, COVID vaccines comprise RNA-loaded lipid nanoparticles, which release the RNA to produce the desired immune response. Multivesicular liposomes deliver payloads in 2-4 days. For example, Exparel, an FDA-cleared multivesicular liposomal bupivacaine formulation can be injected into the surgical site to provide controlled release for up to 72 hours after the operation. PLGA microparticles can release their drug payload in 1-6 months. Zilretta is FDA-cleared for intraarticular injection of 160 mg of PLGA microparticles encapsulating triamcinolone acetonide, which, when released, gives sustained pain relief for 3-4 months. Arestin comprises minocycline hydrochloride loaded PLGA microparticles and is used for infection management in the periodontal pocket. Lupron depot comprises leuprolide acetate loaded PLGA microparticles and is injected intramuscularly to help treat prostate cancer.

PLGA is a bioresorbable (or biodegradable) polymer that can be customized for the desired loading capacity and release kinetics. PLGA bioresorbs or biodegrades in water-based bodily fluids through the hydrolysis of its ester linkages. Several grades of PLGA are commercially available (e.g., Resomer from Evonik) with varying ratios of the monomers, namely lactic acid, and glycolic acid. This ratio and the molecular weight of the resin determine the viscosity and degradation kinetics.

There are several technologies for manufacturing PLGA microparticles and encapsulating therapeutic agents. Hydrophobic compounds can be encapsulated by using single emulsification and solvent evaporation method. Hydrophilic compounds require double emulsion techniques followed by solvent evaporation and lyophilization. This method relies on hydrophilic domains comprising the compounds embedded in the PLGA microparticle matrix. Both the single and double emulsion techniques can be carried out by conventional homogenization methods or by using microfluidic channels to better control particle size distribution and achieve better loading capacity. In the latter method, narrow channels guide the fluid flow with mixing junctions that allow the formation of emulsion droplets. One challenge of the microfluidic method is the undesirable aggregation of microparticles in some cases leading to fiber formation.

### SUMMARY

Controlled release of APIs is desirable in a number of patient treatments and therapies, for instance, wherever a prolonged release would benefit the patient, for instance by reducing the need for multiple doses and for ensuring consistent delivery and presence of an API therapeutically effective amounts. A common use of controlled release dosage forms is in managing and treating pain. Another instance where controlled release of APIs, such as antibiotics, antimicrobials, and other therapeutic agents, is of use is in the prophylaxis or treatment of infections. The present disclosure describes novel dosage forms and related methods of manufacture and use thereof.

In certain embodiments, antibiotic-releasing microsphere technology is administered in an intraarticular injection that can eradicate the infection, reducing the need for surgery. Our technology allows the sustained release of antibiotics above the minimum inhibitory concentration (MIC) and minimum bactericidal concentration (MBC) for weeks at the site of infection. Our technology does not preclude the surgical option and is likely to reduce the likelihood of an infection during open surgery, if it is performed.

In part, the present disclosure describes methods for the encapsulation of various drugs in PLGA microparticles that can be injected directly in or in the vicinity of the site of interest, for instance near an implant where the infection is suspected. The microsphere technology described herein demonstrates prolonged, localized drug delivery and provides a more effective treatment, for example in patients suffering from infections. This approach can eliminate the need for revision surgery in total joint patients, be used prophylactically during surgery by injection into the surgical site or into the surrounding soft and hard tissues, be used to treat suspected infections, and overcome the limits of drug dosage regimes currently used in oral and IV administered drugs.

The present disclosure describes encapsulation in the fabrication of microparticles using microfluidic channel techniques. We discovered that adding a parting channel to a microfluidic chip eliminates aggregation of particles and results in substantial dispersion of microparticles. Increasing the concentration of the stabilizing agent (polyvinyl alcohol) also improved the dispersion of microparticles and reduced the rate of fiber formation.

We also discovered that API(s) can be encapsulated in resorbable polymers using a double emulsion technique, which we enabled in specific microfluidic devices, such as a Y-junction microfluidic device (YJMD), double Y-junction microfluidic device (DYJMD) and double flow-focusing microfluidic device (DFFMD) with and without a parting channel or a Y-junction microfluidic device with a secondary coaxial junction, and others. We also describe here the encapsulation of API(s) in resorbable polymers using a single emulsion technique, which we enabled in specific microfluidic devices, such as a Y-junction microfluidic device (YJMD). Hydrophilic APIs can be encapsulated using double emulsion and hydrophobic APIs can be encapsulated by using the single emulsion technique.

In the present disclosure, we describe several techniques for manufacturing PLGA microparticles and encapsulating active and/or inactive ingredients. In some of these methods, the active and/or inactive ingredients are solubilized in the hydrophilic phase(s). In some of these methods, the active and/or inactive ingredients are solubilized in the hydrophobic phase(s).
Technique 1: We used two YJMDs, one YJMD to make the primary emulsion (PE) where the hydrophilic phase is in the continuous hydrophobic phase (commonly referred to as water in oil or w/o emulsion) and a second YJMD to make the second emulsion where the primary emulsion is in a continuous hydrophilic phase (commonly referred to as water in oil in water or w/o/w emulsion). In this technique, the first water phase and the organic phase are injected into the first YJMD as shown in FIG. 1. The fluid injection may be simultaneous or near simultaneous. The flow direction of respective phases is shown with arrows in FIG. 1. The primary emulsion may form as the two phases flow through the Y-junction. Primary emulsion may then be collected in a container as it exits the first YJMD. The primary emulsion made in the first YJMD may then injected into a second YJMD together or simultaneously or near simultaneously with the second water phase through their respective inlets shown in FIG. 1 (primary emulsion inlet and water phase (w2) inlet. The microparticles may be formed as the flow of the two phases (primary emulsion and second water phase) passes through the Y-junction. The microparticles may be collected in a container as the flow exits the second YJMD outlet. In some instances, the Y junction may have channel geometries, for instance as shown in FIG. 4. These channel geometries may improve the mixing of organic and water phases and/or the primary emulsion and water phases during the formation of either primary and/or secondary emulsions.
Technique 2: We used one DYJMD with three microfluidic channels that we used for injecting the three phases. The injection may be together, for instance simultaneously or near simultaneously, to form the water/organic/water (w/o/w) emulsion. In this technique, the first water phase, the organic phase, and the second water phase are injected into the respective inlets in the DYJMD as shown in FIG. 2 and FIG. 3, where the flow direction of the respective phases is shown with arrows. The primary emulsion forms as the first water phase and the organic phase flows through the Y-junction. The microparticles are formed downstream as the primary emulsion and the second water phase flow through the microfluidic device outlet. The microparticles may be collected in a container. In some instances, the Y junction may have channel geometries as shown in FIG. 4 and as noted these channel geometries may improve the mixing of organic and water phases during the formation of either the primary and/or secondary emulsions.
Technique 3: We used one double flow focusing microfluidic device (DFFMD) with three channels for injecting the three phases, which injection may be simultaneous or near simultaneously to form the w/o/w emulsion. FIGS. 5A and 5B show a schematic of the DFFMD. The flow of the fluids is shown by arrows. The respective solutions are injected through the corresponding inlets as marked on the schematic, including a water phase (w1) inlet, an organic phase (o) inlet and an water phase (w2) inlet. The first flow focusing (FF) junction forms the primary emulsion and the second FF junction forms the secondary emulsion (SE). The microparticles that are formed at and/or after the second FF junction move out of the DFFMD and are collected in a beaker that has an aqueous solution with a surfactant, for instance, PVA in water. One way of controlling the stability of the formed microparticle collected in the beaker is through varying the PVA concentration; higher PVA concentration in most cases will result in better stability of the microparticles and a lower degree of microparticle aggregation.
Technique 4: We used one DFFMD for manufacturing PLGA microparticles with a parting channel as shown in FIG. 6. As shown in FIG. 6, this DFFMD is similar to that shown in FIGS. 5A and 5B for Technique 3. Except for the parting channel, this technique may be similar, the same or nearly the same as Technique 3. The parting channel may be added near the exit of the microfluidic device to dilute the downstream flow with a third water phase, optionally comprising a surfactant such as PVA. The parting channel may have its own parting solution inlet, as shown. The parting channel may increase the water content in the flow and dilute the microparticle concentration, thus decreasing the interaction of the microparticles and decreasing the aggregation of microparticles. The liquid flow exits the microfluidic device at the microparticle outlet, whereby the flow may be collected in a container, such as a beaker. Typically, the beaker has a water solution with or without a surfactant to minimize microparticle aggregation.
Technique 5: In another embodiment we used one YJMD, where the hydrophobic phase forms domains in a continuous hydrophilic phase (commonly referred to as oil in water or o/w emulsion). In this technique, the hydrophobic phase is injected into a YJMD together with the water phase through their respective inlets shown in FIG. 7. The microparticles are formed as the flow of the two phases passes through the Y-junction. The microparticles may be collected in a container as the flow exits the YJMD outlet. This method can be used to encapsulate hydrophobic APIs dissolved in the organic phase by forming a single emulsion of the organic phase in the water phase. For instance, hydrophobic API and resorbable hydrophobic polymer are dissolved in organic solvent to form the organic phase and injected into a YJMD to form microparticles encapsulating hydrophobic API.

In the various techniques described herein, the fluid injection to the inlets may be simultaneous or near simultaneous or may be timed as desired and appropriate to ensure appropriate mixing and formation of microparticles.

In all these techniques the flow of fluid mixture comprising the microparticles may be collected in a container. The container is first filled with an aqueous solution and then fluid exiting the microfluidic device is collected in the said container. The aqueous solution comprises of a surfactant to minimize the aggregation of the microparticles formed.

In all these techniques the organic solvent may be evaporated from the microparticles that are collected from the microfluidic device. This helps in solidifying the microparticles, which may subsequently be lyophilized. The lyophilized microparticles may appear as dry powder and can be injected into the human body by mixing with a dilution fluid.

We formulated microparticles and optimized the fabrication using different PLGA resins. The optimized microparticles resulted in the sustained release of antibiotics for durations up to and exceeding six weeks in laboratory experiments. These microparticles may deliver antibiotics or other active pharmaceutical ingredients locally at therapeutic concentrations to treat infections (for example—S aureus) or provide other therapies, e.g. pain management. The microparticles can be injected subcutaneously, intramuscularly, or intraarticularly at the site of infection, pain management, or other desired location. The microparticles can be customized, for instance release profiles, reabsorption, rates of dissolving by using different types or grades of the bioresorbable polymer, for instance, PLGA with different L/G ratios, or by varying reaction conditions, e.g., pH, temperature, length of reaction time, ratios of ingredients in the reaction mixture, flow rates of reactants, washes.

Overall, this disclosure describes the manufacturing of PLGA microspheres encapsulating active and/or inactive ingredients using single and/or double emulsion microfluidics techniques as well as the use of these microspheres in therapeutic methods.

In comparison with the conventional homogenization techniques used in preparing PLGA microparticles, microfluidic device preparation of microparticles allows (i) increased loading efficiency, that is increased an amount of therapeutic agent encapsulated in the microparticles and (ii) narrower microparticle size distribution, providing better control of elution characteristics, for instance, elution rate and burst release.

Microparticles fabricated with the techniques described above can be combined and administered to achieve desired therapeutic outcomes. For instance, microparticles encapsulating hydrophilic API(s) can be combined with microparticles encapsulating hydrophobic APIs.

In one aspect a method of treating a patient is provided comprising administering to the patient a therapeutically effective amount of an injectable formulation comprising microparticles, and the microparticles comprise at least one active pharmaceutical ingredient and a polymer matrix, for instance a poly(lactic-co-glycolic) acid copolymer (PLGA) matrix, and the microparticles may be manufactured by one or more microfluidic devices.

The microparticles may be manufactured by microfluidic devices using a single emulsion technique. The microparticles may be manufactured by microfluidic devices using a double emulsion method.

The microparticles may comprise at least 5% by weight of one or more infection management active pharmaceutical ingredients or other active pharmaceutical ingredients, for instance, pain management, alleviation or reduction, as well as other therapies.

A microparticle may be formed of a polymer matrix, including a PLGA matrix where the PLGA matrix comprises a lactic acid to glycolic acid molar ratio of 50/50 or 75/25 or of from 50/50 to 75/25.

In certain aspects, the active pharmaceutical ingredient is vancomycin hydrochloride, gentamicin sulfate, rifampicin, or tobramycin, or a combination thereof.

The injectable formulation described herein may comprise a dilution fluid. The injectable formulation described herein may be administered as one or more intra-articular injections.

The formulations described herein may be such that the active pharmaceutical ingredient is released for between at least 7 days and 90 days following administration to a patient.

In certain aspects, the loading capacity of the active pharmaceutical ingredient in the microparticles is above about 5%, from about 5% to 90%, from about 10% to 80%, from about 20% to 60% or from 30% to 50%, by either weight or volume.

The microparticles may further comprise a polyethylene glycol (PEG).

The patient treated with the innovative subject matter described herein may be any of human, animal, plant, microorganism (for instance in or on a substrate or media).

The patient may have a postsurgical infection or be undergoing surgery or the administering may be before the patient undergoes surgery.

A method of making microparticles is disclosed, the microparticles comprising at least one infection management active pharmaceutical ingredient or other active pharmaceutical ingredient and the method may comprise the steps of: delivering an organic phase fluid to a first channel on a microfluidic device, the organic phase comprising a solvent and a polymer material; and delivering a first aqueous phase fluid to a second channel on the microfluidic device, the first aqueous phase comprising water, wherein an active pharmaceutical ingredient is provided in either or both of the organic phase fluid and the first aqueous phase fluid, and the first channel and the second channel on the microfluidic device intersect at a first junction so that the organic phase fluid and the first aqueous phase fluid come in contact to form an emulsion fluid and form microparticles in the fluid, and/or enter a third channel and form microparticles in the fluid; and removing the fluid comprising microparticles from the microfluidic device.

In the methods described herein, the polymer material may be a poly(lactic-co-glycolic) acid copolymer (PLGA) and the solvent may be an organic solvent.

In the methods and microparticles described, the active pharmaceutical ingredient is vancomycin hydrochloride, gentamicin sulfate, rifampicin, or tobramycin, or any combination thereof.

The methods may comprise separating the microparticles from the fluid and washing the microparticles to remove the solvent. Any suitable separation techniques may be use, for instance separation by density, for instance, centrifuging, or by size, for instance, with filtration.

The methods may also comprise the step of drying the microparticles. Drying may include the application of heat, cold (freezing), evaporation (including providing a dry environment to encourage evaporation). The step of drying may comprise lyophilizing the microparticles.

The active pharmaceutical ingredient may be provided in only the organic phase fluid. Alternatively, or additionally, the active pharmaceutical ingredient may be provided in only the first aqueous phase fluid or in both the organic phase fluid and the aqueous phase fluid.

The method may comprise delivering a second aqueous phase fluid to a fourth channel, and the fourth channel may have a second junction, where the second junction intersects the third channel. The second junction and fourth channel may be on a second microfluidic device or the second junction and fourth channel are on the same microfluidic device as the first junction and first channel.

In the methods, the second aqueous phase fluid may comprise any or all of water, an alcohol, polyvinyl alcohol and/or a salt or salt solution.

The methods described herein may comprise delivering the fluid comprising microparticles into a first channel on a second microfluidic device and delivering a second aqueous phase fluid to a second channel on the second microfluidic device.

In one embodiment the microparticles may have about 6% of active pharmaceutical ingredient by weight, or about 5-80%, 10-70%, 20-60%, 30-50%, or 40% by weight or volume. The microparticles may have multiple active pharmaceutical ingredients.

In one embodiment the microparticles have a molecular weight in the range of about 20-80 kDa, 30-70 kDa, 40-60 kDa, 50 kDa.

In one embodiment, the microparticles have a lactic acid: glycolic acid molar ratio of 50:50 to 75:25 or in a range of from 75:25 to 75/25 or 50:50.

The microparticles may have a viscosity of 0.15 to 0.60 dL/g, from 0.2 to 0.5 dL/g, from 0.3 to 0.4 dL/g. These viscosity parameters may also apply to a fluid containing the microparticles.

The microparticles may have a mean diameter of from or between 10 to 50 μm, 20 to 40 μm, or 30 μm.

The methods and the microparticles may release the active pharmaceutical ingredient for a time period of up to 4-6 weeks or longer following injection into a patient or when placed in an in vitro media bath or substrate at physiologic conditions, for instance any of pH, temperature, etc.

The flow rate of the emulsion comprising microparticles is in the range of 4-18 ml/min, 6-16 ml/min, 8-14 ml/min, 10-12 ml/min.

The ratio of aqueous phase fluid to organic phase fluid may be about 9:1 or in a range of from 1:9 to 9:1, including 1:1, 8:1, 6:1, 4:1, 3:1 or 1:8, 1:6, 1:4, or 1:3.

The method may further comprising putting the emulsion in contact with a second aqueous phase fluid where the ratio of emulsion to second aqueous phase fluid is about 1:6 or in a range of from 1:9 to 9:1, including 1:1, 8:1, 6:1, 4:1, 3:1 or 1:8, 1:6, 1:4, or 1:3.

Also contemplated are microparticles produced by the methods described herein.

In another aspect, a high throughput microfluidic device for producing emulsions is provided, comprising: at least one microchannel connected to a flow source, the microchannel having a completely or partially void structure to form a cavity, the cavity allowing fluids, dispersions and/or emulsions to pass through the cavity, wherein at least one of the width, length or height of the channel is a maximum 1000 micrometers, or is in a range of 100-10,000 micrometers, or 400-5,000 micrometers, or 600-3000 micrometers, and at least two fluid inlets are connected to the microchannel and allow fluid to enter the microchannel, and at least one fluid outlet is connected to the channel and enables the fluid to exit from the microchannel, at least two flow focusing junctions are located between the fluid inlet and the fluid outlet in the channel, which changes the flow properties of the fluid in the channel to form emulsions in the channel, or in one or more of the junctions.

The microfluidic devices described may be high throughput microfluidic devices.

The microfluidic devices may have at least three fluid inlets connected to a microchannel.

The microfluidic devices may have two flow focusing junctions connected to the microchannel.

The microfluidic devices may have a microchannel connect the fluid inlets and the fluid outlet, and one or more flow focusing junctions may be placed into the microchannel.

The microfluidic devices may have microchannels made of glass or polymetalmethacrylate (PMMA) material, or other suitable material that is preferably inert.

### BRIEF DESCRIPTION OF THE DRAWING

Some embodiments of the disclosure are described herein with reference to the accompanying figures. The description, together with the figures and Appendix attached hereto, make apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. Additional objects, advantages and novel features of the present disclosure will become apparent from the following detailed description, particularly when considered in conjunction with the accompanying drawings.

FIG. 1 shows two Y-junction microfluidic devices (YJMD). Each microfluidic device has two inlets for injecting different phases and one outlet. Primary emulsion is formed in the first device, collected from the PE outlet in a container, and injected into the second device with w2 to form PLGA microparticles.

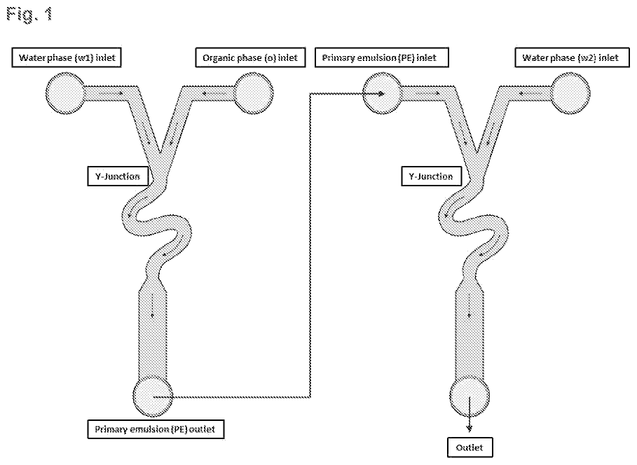

FIG. 2 shows a double Y-junction microfluidic device (DYJMD) is shown here. This device has three inlets for the o, w1, and w2 phases. The primary emulsion is formed at the Y-junction, and the secondary emulsion is formed at the outlet.

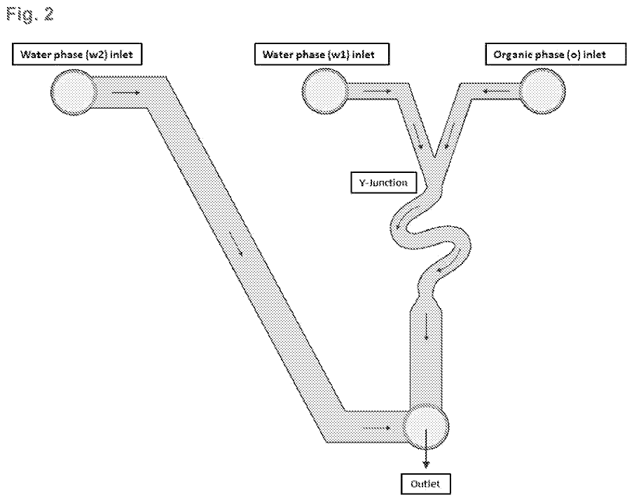

FIG. 3 shows a double Y-junction microfluidic device (DYJMD) is shown here. This device has three inlets for the o, w1, and w2 phases. The primary emulsion is formed at the Y-junction. The secondary emulsion is formed at the junction where the PE merges into w2 phase and forms PLGA microparticles, before the flow reaches the outlet.

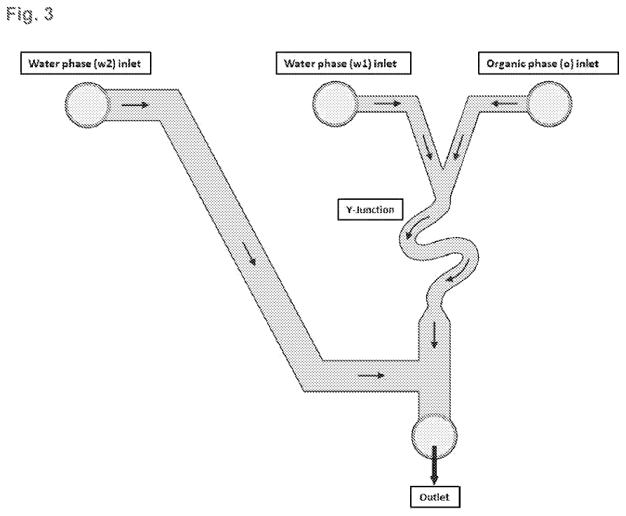

FIG. 4 shows a Y junction microfluidic device (or may be used to envision a portion of a device) similar to that of FIGS. 1, 2, and 3 with channel geometries as shown. These channel geometries are structures, for instance with additional curves, passages points of separation and junction, to improve the mixing of organic and water phases during the formation of either primary and/or secondary emulsions.

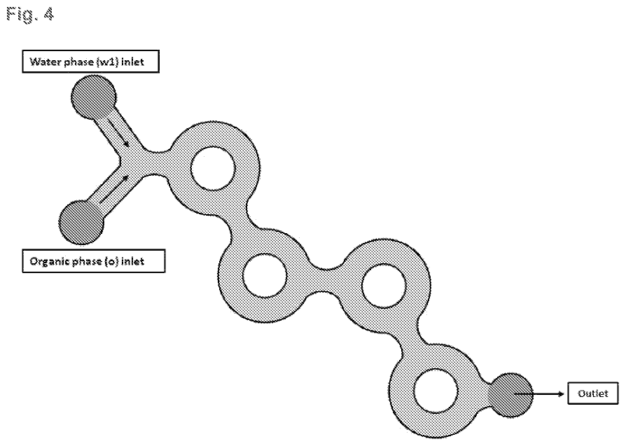

FIGS. 5A and 5B show a double flow focusing junction microfluidic device (DFFMD). The microfluidic device has three inlets for the three different phases and one outlet for double emulsion droplets as shown in FIG. 5A. An exploded view of the first and second flow focusing junctions in FIG. 5A are shown in 5B. Primary and secondary emulsions are formed as the flow passes through the first and second flow focusing junctions, respectively.

FIG. 6 shows a double flow-focusing junction microfluidic device (DFFMD) with a parting channel. The microfluidic device has four inlets for the four different phases and one outlet. This device is like the one shown in FIG. 3 with the added parting phase inlet.

FIG. 7 shows a single Y-junction microfluidic device (YJMD). The microfluidic device has two inlets for different phases and one outlet. The emulsion is formed in the chip at the Y junction and may be collected from the outlet in a container.

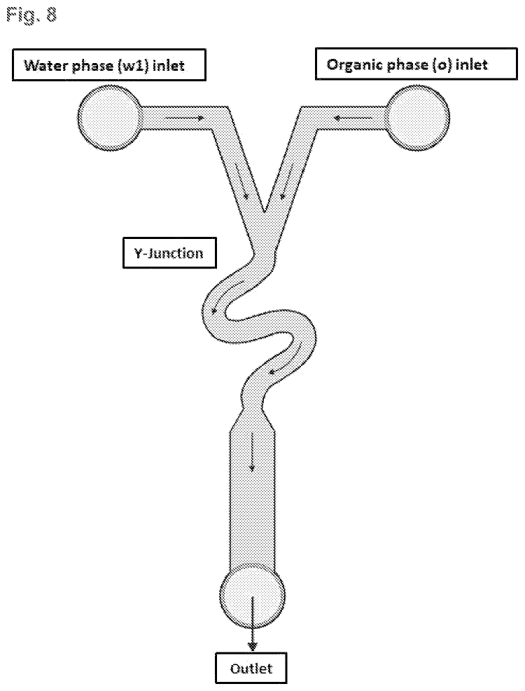

FIG. 8 shows an example of a UV absorbance calibration curve for vancomycin hydrochloride in PBS using a UV-Visible plate reader. Solutions with known concentrations of vancomycin hydrochloride in PBS are prepared and their UV absorbances are measured at 280 nm. These absorbances are plotted as a function of corresponding concentrations. The linear regression shown is used to determine the vancomycin hydrochloride concentration in a solution of unknown concentration. A new calibration is prepared for each experiment where elution rate or the loading capacity are measured.

FIG. 9 shows transmission optical microscopy images of F1 PLGA microparticles comprising vancomycin hydrochloride of Example 4. Microparticles were imaged after the washing step as described in Example 4. The scale bar is 50 μm for panels A and B and 20 μm for panels C and D.

FIG. 10 shows transmission optical microscopy images of F2 PLGA microparticles comprising vancomycin hydrochloride of Example 4. Microparticles were imaged after the washing step as described in Example 4. The scale bar is 50 μm for panels A and B and 20 μm for panels C and D.

FIG. 11 shows a graph of the percent cumulative elution of vancomycin hydrochloride from the formulation F1, vancomycin hydrochloride loaded PLGA microparticles, of Example 4. Percent cumulative vancomycin elution was determined by the method described in Example 3.

FIG. 12 shows a graph of the percent cumulative elution of vancomycin hydrochloride from the formulation F2, vancomycin hydrochloride loaded PLGA microparticles, of Example 4. Percent cumulative vancomycin elution was determined by the method described in Example 3.

FIG. 13 shows transmission optical microscopy images of the formulation F3, PLGA microparticles comprising vancomycin hydrochloride, of Example 5. Microparticles were imaged after the washing step as described in Example 5. The scale bar is 50 μm for panels A and B and 20 μm for panels C and D.

FIG. 14 shows a graph of the percent cumulative elution of vancomycin hydrochloride from F3 vancomycin hydrochloride loaded PLGA microparticles of Example 5. Percent cumulative vancomycin elution was determined by the method described in Example 3.

FIG. 15 shows transmission optical microscopy images of F4 PLGA microparticles comprising vancomycin hydrochloride of Example 6. Microparticles were imaged after the washing step as described in Example 6. The scale bar is 200 μm for all panels A, B, C and D.

FIG. 16 shows transmission optical microscopy images of F5 PLGA microparticles comprising vancomycin hydrochloride of Example 7. Microparticles were imaged after the washing step as described in Example 7. The scale bar is 100 μm for panel A and 50 μm for panel B.

FIG. 17 shows a graph of the percent cumulative elution of vancomycin hydrochloride from F5 vancomycin hydrochloride loaded PLGA microparticles of Example 7. Percent cumulative vancomycin elution was determined by the method described in Example 3.

FIG. 18 shows transmission optical microscopy images of F6 PLGA microparticles comprising rifampicin of Example 8. Microparticles were imaged after the washing step as described in Example 8. The scale bar is 50 μm for panels A and B and 20 μm for panels C and D.

FIG. 19 shows a timeline comparison of treatment techniques for a knee, the first technique involving multiple surgeries and the second technique involving a single surgery with an intraarticular injection of API containing microspheres as described herein.

FIG. 20 show a sequence of steps in a method for making microspheres containing API in accordance with certain embodiments of this disclosure.

### DETAILED DESCRIPTION

For simplicity and illustrative purposes, the principles of the present invention are described by referring to various example embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. It is contemplated that the various features described herein may be combined and/or excluded in any number of different combinations, all of which are part of the inventive contribution set forth herein. The features and particulars of the microparticles, their methods of use and methods of manufacture are intended to be understood to apply to each of the microparticles themselves, their methods of use and their methods of manufacture, even though, for instance a particular property may be described as applying to the microparticles, their methods of use or of manufacture. The terminology used herein is for the purpose of description and not of limitation.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” As used herein, the term “about” includes values up to and down to 5% of a stated value. As used herein, any described ranges include the uppermost and lowermost stated value, as well as every whole number in between. So, for instance, it is contemplated that a range of 1 to 4 expressly includes each of the values 1, 2, 3, and 4.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Microparticles created in accordance with the teachings of this disclosure and related therapeutic methods and methods of manufacture are provided. Microparticles are particles which may or may not be spherical in morphology, e.g., microspheres. Microparticles may encapsulate one or more APIs and/or other inactive ingredient(s). Microparticles may be fabricated from polymeric material(s). The polymeric material may typically be resorbable which facilitates the release of encapsulated ingredient(s). For example, microparticles may be fabricated with poly(lactic-co-glycolic) acid copolymer (PLGA) using single emulsion and/or double emulsion techniques. The PLGA in microparticles may be a blend of different resin grades with varying molecular weights, varying viscosities, and/or varying ratios of lactic acid to glycolic acid.

API means active pharmaceutical ingredient. API may be a single active pharmaceutical ingredient or a mixture of active pharmaceutical ingredients. For instance, vancomycin, tobramycin, gentamicin, cefadroxil, cefazolin, cephalexin, cefaclor, cefotetan, cefoxitin, cefprozil, cefuroxime, loracarbef, cefdinir, cefixime, cefoperazone, cefotaxime, cefpodoxime, ceftazidime, ceftibuten, ceftozoxime, ceftriaxone, cefepime, amikacin, streptomycin, doxycycline, erythromycin, gentamicin, isoniazid, rifampin, and ethambutol. Sulfonamides, beta-lactams including penicillin, cephalosporin, and carbepenems, aminoglycosides, quinolones, and oxazolidinones, and metals such as copper, iron, aluminum, zinc, gold, compound, and ions thereof, and various combinations thereof are contemplated. Lipopolysaccharides (LPS), polyguanidines (CPG), bacterial lysates, defensins and their salts such as hydrochloride sodium, sulfate, acetate, phosphate or diphosphate, chloride, potassium, maleate, calcium, citrate, mesylate, nitrate, tartrate, aluminum, and/or gluconate are also contemplated. For instance, vancomycin hydrochloride, gentamicin sulfate, tobramycin sulfate, and/or polyhexamethylene guanidine phosphate or mixtures thereof are also contemplated.

By infection management API is meant antibiotics including but not limited to vancomycin, tobramycin, gentamicin, cefadroxil, cefazolin, cephalexin, cefaclor, cefotetan, cefoxitin, cefprozil, cefuroxime, loracarbef, cefdinir, cefixime, cefoperazone, cefotaxime, cefpodoxime, ceftazidime, ceftibuten, ceftozoxime, ceftriaxone, cefepime, amikacin, streptomycin, doxycycline, erythromycin, gentamicin, isoniazid, rifampin, ethambutol, and their various salts. Sulfonamides, beta-lactams including penicillin, cephalosporin, and carbepenems, aminoglycosides, quinolones, and oxazolidinones, and metals such as copper, iron, aluminum, zinc, gold, compound, and ions thereof, and various combinations thereof are also contemplated. Lipopolysaccharides (LPS), polyguanidines (CPG), bacterial lysates, defensins and their salts such as hydrochloride sodium, sulfate, acetate, phosphate or diphosphate, chloride, potassium, maleate, calcium, citrate, mesylate, nitrate, tartrate, aluminum, and/or gluconate are also contemplated. For instance, vancomycin hydrochloride, gentamicin sulfate, tobramycin sulfate, and/or polyhexamethylene guanidine phosphate or mixtures thereof, antimicrobials, antimicrobial peptides, including but not limited to non-steroid anti-inflammatory drugs (NSAIDs), and analgesics are also contemplated.

In some embodiments the microparticles comprise inactive ingredients. By inactive ingredient is meant substances that have no known therapeutic effects. Some inactive ingredients are added to the solutions used in the microfluidic fabrication of the microparticles to increase the loading capacity of the active ingredient in the resulting microparticles. Some inactive ingredients assist in controlling the release kinetics of the ingredients from the microparticles, the storage stability, the need for cold, frozen or refrigerated temperatures. These inactive ingredients include but are not limited to viscosity modifiers, salts, pH modifiers, surfactants, solvents, and/or gases or mixtures thereof.

The microparticles are injected into the human body for therapeutic effect(s); therefore the microparticles need to be injectable. In some embodiments an injectable microparticle formulation is microparticles in a dilution fluid. The injectable microparticle formulation may comprise a suspension, an emulsion, and/or a dispersion in a dilution fluid together with microparticles. In certain embodiments the injectable microparticle formulation comprises more than one type of microparticles that encapsulated different API.

By dilution fluid is meant the solution comprising any of viscosity modifier(s), surfactant(s), buffer(s), salt(s), pH modifier(s), and/or solvent(s) or mixtures thereof. The microparticles are typically mixed with the dilution fluid and injected into the human body, for example into the joint space, soft tissue, muscle, fat tissue or others desired site.

By viscosity modifier(s) is meant polyvinylpyrrolidones (PVP) (preferably having a molecular weight of about 10,000 or less to about 360,000 or more, or from 50,000 to 300,000, or from 100,000 to 200,000, as well as mixtures comprising one or more grades of PVP with different molecular weight, cellulose derivatives (including, but not limited to, hydroxyethyl cellulose, carboxymethyl cellulose or its salts, hypromellose, and the like), glycosaminoglycans including but not limited to heparin, chondroitin sulfate, keratan sulfate, heparan sulfate or their salts, carrageenan, guar gum, alginates, carbomers, polyethylene glycols, lipids, oils, sugars, polyvinyl alcohol, xanthan gum, and/or their derivates or mixtures thereof. In certain preferred embodiments, carboxymethyl cellulose is used as a viscosity-increasing agent in the microparticles formulations provided by the present invention. Viscosity modifier(s) may be added to the dilution fluid.

In certain embodiments, the polymer or polymers may be cross-linked or treated, for instance with UV activation.

As used herein, the term about means a range above and below the stated value by 5%.

Surfactants, including dispersants, may be used to minimize the agglomeration of microparticles. The surfactants are nonionic, anionic, cationic, and/or amphoteric. Examples of nonionic surfactants that can be used in the present invention are poly(vinyl alcohol) (PVA), poloxamer 188, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), and/or polyoxyethylene nonylphenol ether (Nonoxynol®) and lecithin) or mixtures thereof. Examples of anionic surfactants that can be used in the present invention are ammonium lauryl sulfate, sodium laureth sulfate, sodium lauryl sarcosinate, sodium myreth sulfate, sodium pareth sulfate, sodium stearate, sodium lauryl sulfate, a olefin sulfonate, and/or ammonium laureth sulfate or mixtures thereof. Examples of cationic surfactants that can be used in the present invention are benzalkonium chloride, and/or cetylpyridinium chloride or mixtures thereof. Examples of amphoteric surfactants that can be used in the present invention are betaines or sulfobetaine and natural substances such as amino acids and/or phospholipids or mixtures thereof. One of the preferred surfactants used in the present invention is poly(vinyl alcohol).

Salts used in this invention may include but are not limited to sodium chloride, calcium chloride, citrates, acetates, potassium dihydrogen phosphates, disodium hydrogen phosphates, or mixtures thereof.

The pH modifiers are substances which can either increase or decrease the pH of the solution they are added to, or, for instance, act as a buffer. Examples are soda ash, sodium hydroxide, sodium silicate, sodium phosphates, lime, sulfuric acid, hydrofluoric acid, tri-potassium citrate monohydrate, sodium hydrogen carbonate, tartaric acid, calcium carbonate, and/or adipic acid or mixtures thereof.

Solvents used in his invention can be an organic solvent or an aqueous solvent. Examples of aqueous solvents include but are not limited to sterile water, phosphate buffer, or saline solution. Examples of organic solvents include but are not limited to DMSO, ethyl acetate acetonitrile, N-methyl pyrrolidone, chloroform, and/or dichloromethane or mixtures thereof. The solvents are used to prepare solutions or phases used in the microfluidic devices to fabricate microparticles.

By dispersion agent or emulsifying agent is meant a surfactant that helps the formation of emulsion droplets and stabilizes the emulsion. Dispersion agent(s) aid in inhibiting the aggregation of microparticles. The dispersion agent(s) can be used in the first water phase, second water phase, and/or the parting solution. For example, PVA can be added to water to act as a dispersion agent.

By first water phase (w1) is meant an aqueous solution comprising water, deionized water, PBS and/or other aqueous solvents in which at least one API and/or inactive ingredient is solubilized. The first water phase can also comprise a mixture of various aqueous solvents, such as water, deionized water, and/or PBS as well as surfactants. The first water phase can also comprise added salts such as sodium chloride (NaCl). Concentration of salt in first water phase can be about 0.5M NaCl solution or more than 0.5M NaCl or less than 0.5M NaCl. The first water phase can also comprise one or more pH modifiers.

Organic phase (o) comprises polymeric material(s) and/or API or their combinations solubilized in organic solvents such as ethyl acetate, acetonitrile, N-methyl pyrrolidone, chloroform, and/or dichloromethane or mixtures thereof. For example organic phase may comprise PLGA solubilized in ethyl acetate.

The second water phase (w2) is an aqueous solution comprising dispersion agent(s), such as poly vinyl alcohol (PVA) or other surfactants, and/or inactive ingredient(s) solubilized in water, deionized water or PBS or other aqueous solvents. The second water phase can also have added salt(s) such as sodium chloride, and/or calcium chloride. In some embodiments the second water phase also has API(s) added. In one example, the concentration of salt (NaCl) in the second water phase is 0.5M.

By water phase (w) is meant an aqueous solution comprising dispersion agent(s), such as poly vinyl alcohol (PVA) or other surfactants, and/or inactive ingredient(s) solubilized in water, deionized water or PBS or other aqueous solvents. The second water phase can also have added salt(s) such as sodium chloride, and/or calcium chloride. In some embodiments, the second water phase also has API(s) added. In one example, the concentration of salt (NaCl) in the water phase is 0.5M.

By aqueous solution is meant a solution in which solutes such as dispersion agent(s), for example PVA, and/or API are solubilized in water, ionized water or PBS or other aqueous solvents. Aqueous solution may comprise salts such as sodium chloride, calcium chloride, citrates, acetates, potassium dihydrogen phosphates, disodium hydrogen phosphates, or mixtures thereof.

Primary emulsion (PE or w1/o) comprises first water phase (w1) emulsion particles dispersed in organic phase (o).

Secondary emulsion (SE or w1/o/w2) comprises primary emulsion dispersed in second water phase (w2). The secondary emulsion is also referred to as double emulsion. Secondary emulsion comprises emulsion particles of the organic phase. The organic phase emulsion particles comprise w1 emulsion particles. In certain embodiments, secondary emulsion may comprise oil emulsion droplets in water, where the oil emulsion droplets comprise water droplets. Said water droplets may comprise API. The secondary emulsion may be subjected to solvent dehydration at room temperature or at lower and/or higher temperatures to partially or fully remove the organic and/or the aqueous solvent. The solvent dehydration of the double emulsion results in microparticles. In some embodiments the dispersion agent, such as PVA, may be fully or partially removed by wash cycle(s). In some embodiments the microparticles may be dried or lyophilized to partially or fully remove water. Suitable drying methods may include heat, freezing, evaporation and/or combinations thereof.

Single emulsion (o/w) comprises organic phase dispersed in water phase (w). Single emulsion may be subjected to solvent dehydration at room temperature or at lower and/or higher temperatures to partially or fully remove the organic and aqueous solvent. The solvent dehydration of the single emulsion may result in formation of microparticles. Optionally, the dispersion agent is removed by wash cycle(s), and/or the microparticles are dried or lyophilized to remove water from the microparticles, for instance, PLGA microparticles.

Polymeric material can be a resorbable polymer such as poly(lactide-co-glycolide) (PLGA), polylactide (PLA), polyglycolide (PG), polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), polyurethanes, polythioesters, poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, and PLGA-PEO-PLGA blends and copolymers thereof and any combinations thereof, or lipids such as phosphatidylcholines, phosphatidylethanolamines, phosphatidylglycerols, phosphatidic acids, phosphatidylinositols, and/or sphingomyelins or mixtures thereof.

Poly lactic-glycolic acid polymer or PLGA is a polymer that comprises repeat units of glycolic acid and lactic acid. The molecular weight of PLGA used in this invention is in the range of about 20-80 kDa or below 20 kDa or above 80 kDa. PLGA used in this invention may have a lactic acid to glycolic acid molar ratio ranging from 1:99 to 99:1, about 25:75, about 50:50 to about 75:25. PLGA may have a viscosity of about 0.15 to 0.60 dL/g in chloroform. The PLGA may be acid or ester terminated. The PLGA is a blend of acid or ester terminated resins. One commercially available PLGA resin is Resomer, and it is supplied by Evonik. RESOMER® RG is a platform of PLGA/poly (D, L-lactide-co-glycolide)-based bioresorbable excipients for controlled release. Designed for use with a range of complex parenteral drug products, these amorphous polymers are also available with either acid or ester end groups. Standard mole ratios include 50:50, 65:35, 75:25 and 85:15. Degradation times can extend for up to 18 months or more. Inherent Viscosities (IV) can range from 0.09 to 1.7 dL/g, with molecular weights between 7,000 and 240,000 Daltons. Polymer properties including crystallinity can be tuned to match specific formulation and release profile requirements. Resomer for parenteral products are named using a specific naming convention. For example, Resomer 503 h is named as follows: H indicates acid termination, 50 indicates lactide concentration, that is a lactide: glycolide ratio of 50:50, 3 indicates the inherent viscosity of the Resomer is 0.32-0.44 dL/g, 0.1% (w/v) in chloroform (25° C., Ubbelohde, size OC glass capillary viscometer). Some other manufacturers of PLGA resin include Ashland (Viatel) and Corbion (Polysorb). In this invention any commercial PLGA resin or their blends can be used to fabricate the described microparticles.

By loading capacity is meant the amount of API(s) in milligrams found in 100 milligrams of microparticles. For instance, 10% loading capacity means 10 mg of API encapsulated in 100 mg of microparticles. Suitable loading capacities may be 5%, or may be ranges from 5% to 90%, or from 10% to 80%, from 20% to 70%, from 30% to 60% or any combination of these range values.

By PVA is meant poly vinyl alcohol. PVA can have a weight average molecular weight of less than 3,000 Da, about 3,000-23,000 Da, or above 23,000 Da. PVA is typically 87-89% hydrolyzed or less than 87% hydrolyzed or more than 89% hydrolyzed. PVA is a mixture of PVAs with different weight average molecular weights and/or hydrolyzation levels.

By parting solution is meant a solution used to prevent the aggregation of microparticles in the microfluidic device. The parting solution may comprise a surfactant such as PVA in varying concentrations. For instance, the parting solution may be an aqueous solution of PVA. Or the parting solution may be an aqueous solution of PVA with NaCl. In some embodiments the parting solution may be the same as the second water phase or the first water phase. Parting solution may comprise dispersion agent(s), such as poly vinyl alcohol (PVA), and/or inactive ingredient(s) solubilized in water, deionized water or PBS, or other aqueous solvents, viscosity modifier(s), surfactant(s), buffer(s), salt(s), pH modifier(s), and/or solvent(s) or mixtures thereof. In some embodiments, solvents include methanol, ethanol, propanol, hexane, pentane, heptane, and dichloromethane and may be added to the parting solution. In some embodiments, the second water phase also has API(s) added. The concentration of salt in second water phase can be about 0.5M NaCl solution or a concentration in a range from 0.1 to 2M NaCl or from 0.3 to 1M NaCl.

By microchannel is meant a flow channel in a microfluidic device. Microchannels may connect inlets to each other or to an outlet. The cross-sectional shapes of the microchannel can be square, rectangle, round, or other complex shapes. Suitable materials for the microchannels may be glass or polymetalmethacrylate (PMMA) material, or other suitable materials.

By inlet is meant the openings where fluid is provided to and enters the microfluidic device. Some inlets enable syringe pumps to pump the fluids into the microfluidic device with a predetermined flow rate. Said fluid may be the first water phase, second water phase, organic phase, parting solution, or water phase used in the fabrication of primary or secondary emulsions.

By outlet is meant the openings where fluid exits the microfluidic device. The outlets enable any fluid to leave the microfluidic device.

By junction is meant a connection between or joining to or more microchannels. The junctions can be in T shape, Y shape, or + shape. The + shape junctions are also referred to as flow-focusing junctions.

By flowrate is meant the volume of fluid flow per unit time. For instance, the flowrates at which the organic phase, the first and second water phases, and the parting solution are injected into the microfluidic devices. These flowrates may be varied to optimize the microparticle size distribution and API(s) loading capacity. The flowrates are typically controlled by syringe pumps.

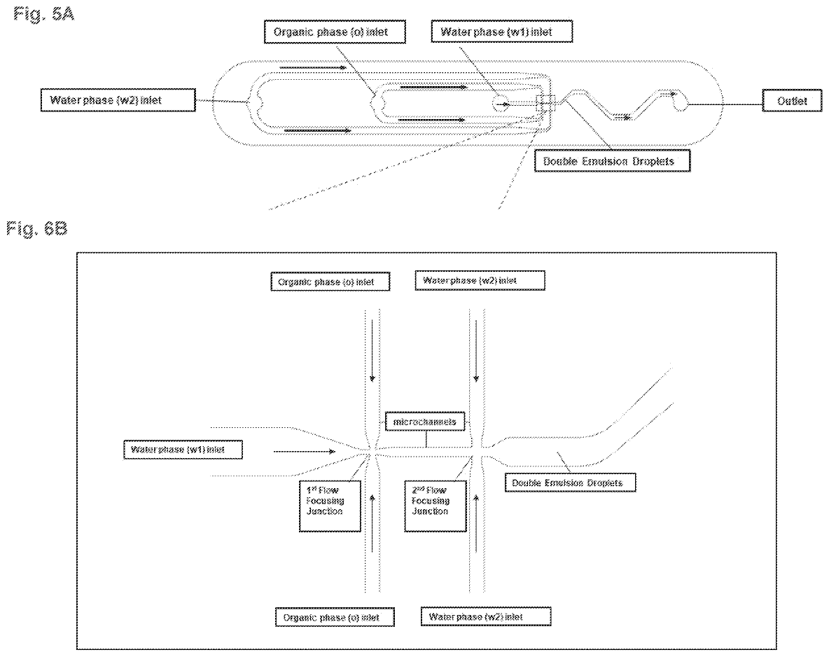

By a high throughput microfluidic device (FIG. 5A) is meant a microfluidic device used to produce multiple emulsions. A microfluidic device has at least one microchannel (FIG. 5B) which allows solutions, dispersions, or emulsions to pass through the said microchannel together with the fluid and has a completely or partially void structure to form a channel where at least one of the width, length, or height of the microchannel is a maximum of 1000 micrometer. A microfluidic device has at least two inlets (FIG. 5A) that connect the microchannel (FIG. 5B) to a flow source and allow the fluid to enter the microchannel. A microfluidic device has at least one outlet (FIG. 5B) connected to the microchannel and enables the fluid to exit from the microchannel. A microfluidic device has at least one junction, for instance, a flow-focusing junction (FIG. 5B), located between the inlet and the outlet. The said junction helps in forming an emulsion(s) in the microchannel (FIG. 5A). A microfluidic device is made of glass or polymethylmethacrylate (PMMA) material, 3D printable polymers, PEEK, photocurable polymers, or other materials.

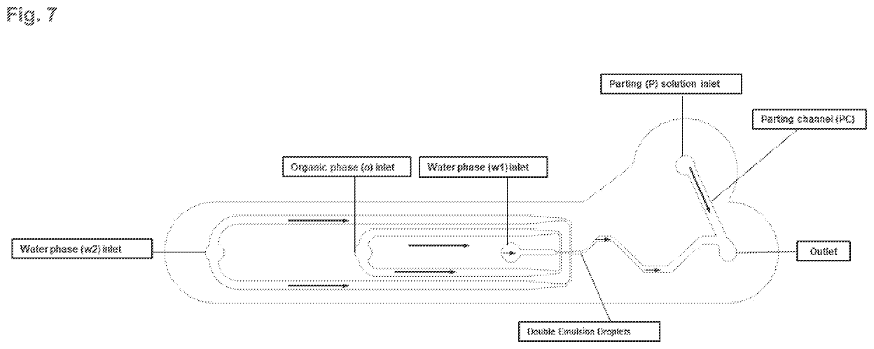

A parting channel is shown in FIG. 6, as a channel is provided on the microfluidics device to allow addition of a fluid, for instance near the outlet. The parting channel enables the parting of droplets or nascent particles from each other with a parting solution at a certain flow rate. Parting the droplets or nascent particles also means to disperse the droplets or nascent particles, and/or to minimize the aggregation of the droplets or nascent particles.

The terms ‘phase’ and ‘solution’ may be used interchangeably. For instance, water phase also means water solution.

In one embodiment, PLGA microparticles (or microparticles of other polymer material) are produced and therapeutic agent(s) are encapsulated in these microparticles with a simultaneous double emulsion technique in a microfluidic system.

In another embodiment, the microfluidic system is extended from double emulsion to multiple emulsions.

In some embodiments, a parting channel and a parting solution input may be provided on the microfluidic device and may serve to minimize microparticle or emulsion droplet aggregation. A gradual and/or proportional increase in the dimensions of the channels at the point of flow-focusing junction enables increased loading capacity for API encapsulation and an additional parting channel prevents microparticle aggregation. With this high throughput microfluidic design, it is possible to feed a single microfluidic device with a flowrate up to a liter per hour or more to fabricate PLGA microparticles. The parting channel may also reduce the amount of surfactant(s) or stabilizer(s) used in the microfluidic device.

Treatment with Microspheres

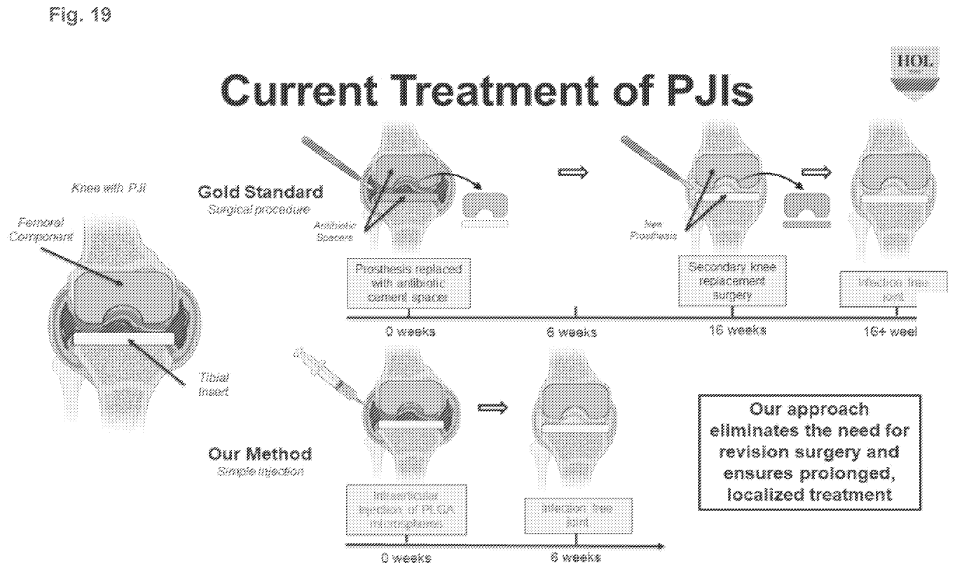

FIG. 19 shows a timeline comparing treatment techniques for a knee. The upper timeline, extending over 16+ weeks, shows a surgery to remove a prosthesis from an earlier knee replacement surgery, and replace the prosthesis with an antibiotic cement spacer. Then, 16 weeks later, the patient undergoes another replacement knee surgery, this one to remove the antibiotic cement spacer and install a traditional prosthesis. The bottom panel shows a timeline for injection of microspheres containing active pharmaceutical ingredients as disclosed herein. microspheres containing active pharmaceutical ingredients are injected into the knee and the active ingredient, for instance, an antibiotic, can be released over time (extended or prolonged release) to work as a therapeutic effective to treat, prevent, and/or inhibit infection.

### Manufacture of Microspheres

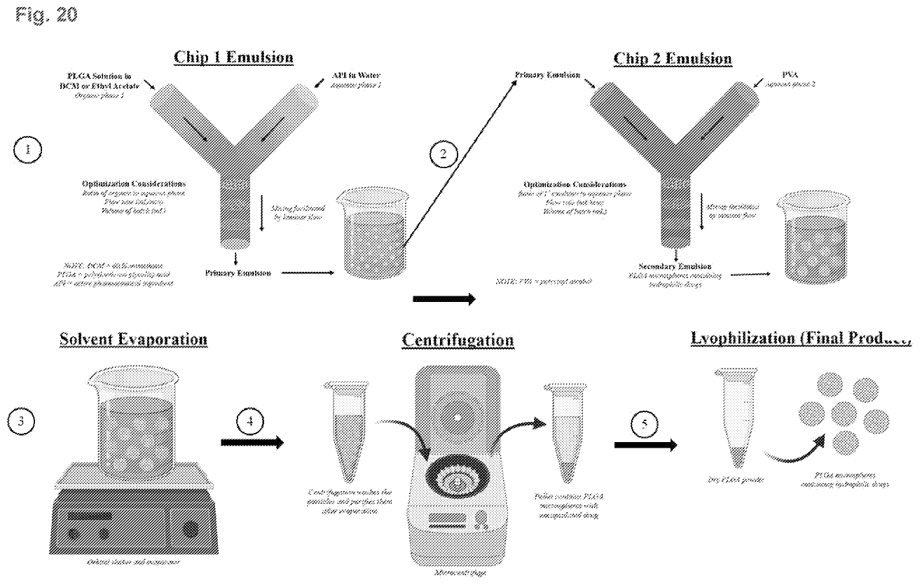

FIG. 20 show a sequence of steps in a method for making microspheres containing API in accordance with certain embodiments of this disclosure. In this embodiment, a polymer precursor solution, PLGA solution in DCM or ethyl acetate are provided as an organic phase at an organic phase inlet and the active pharmaceutical ingredient in water is added at a water phase inlet of a microfluidic device. Channels from each inlet merge at a junction where the organic and aqueous phases mix. Mixing of the organic and aqueous phases may be improved by providing structure to encourage laminar or turbulent flow, as desired. A primary emulsion may be recovered from the microfluidic device and this primary emulsion may be added to a second microfluidic device (or a separate portion of the same microfluidic device) at a primary emulsion inlet while a second aqueous phase is added at a second aqueous phase inlet. The second aqueous phase may include PVA. Channels from each inlet merge at a junction where the primary emulsion phase and second aqueous phases mix. PLGA microspheres form in fluid and the microspheres contain the active ingredient. The fluid and microspheres may be recovered as a secondary emulsion.

Mixture parameters of the fluids may be optimized for the ratio of multiple phases being mixed, the flow rate, and the batch volume, as well as other desired parameters, such as pH, temperature, pressure, laminar or turbulent flow, etc.

The microspheres may be removed from the fluid, for instance, by evaporation of solvents, centrifuging and washing the microspheres. Additionally, the washed microspheres may be dried to form a dry lyophilized product of microspheres. Preferably the dry microspheres are stable at room temperature and may be stored before being mixed into a liquid for delivery to a patient, for instance by injection.

With a view to attain sustained release of antibiotics in the joint space, we developed PLGA microspheres using PRECISION NANO systems microfluidics system. The manufactured microspheres are loaded with antibiotics to produce the antibacterial effect at the site of injection. Alternatively other active pharmaceutical ingredients may be added instead of or in addition to antibiotics. These microspheres were shown to deliver antibiotics at the predetermined rate for at least 6 weeks at the concentration above the MIC and MBC of the organism associated with the postsurgical infection (for example—S aureus). The microspheres can be injected intraarticularly, intramuscularly or intraarticularly at the site of infection. The formulation can be customized by using different grades of polymer with L/G ratios ranging from 50:50 to 75:25 by double emulsion solvent evaporation technique with varying amount of antibiotics to produce the antibacterial effect.

Intermittent double emulsion technique: PRECISION NANOSYSTEM (PN) is a microfluidic system that utilizes a single microfluidic (MF) device to mix two fluids at one mixing junction. We used the PN system to fabricate microparticles with double emulsion method in two steps. We formed the first emulsion by injecting the water/API solution and a PLGA/organic solvent mixture through the device and collected the first emulsion. We then injected the first emulsion into the PN device together with PVA/water solution to form the second emulsion. The first emulsion had water/API droplets in the organic phase and the second emulsion had water/API droplets encapsulated in PLGA/organic phase droplets where these PLGA/organic phase droplets were dispersed in PVA/water solution as shown in FIG. 20.

Simultaneous double emulsion technique: In another aspect we provide a high throughput multiple emulsion microfluidic device which is shown in the accompanying figures, for instance FIG. 5 shows a view of a high throughput multiple emulsion microfluidic device, having microchannels, fluid inlets, a fluid outlet, and flow focusing junctions. The inventive principles described herein would be operative on different MF devices including those with more than one mixing junction and fabricated microparticles using double emulsion.

### Embodiment A: Double Emulsion with Two Intermittent Consecutive Single Emulsion Steps

In one embodiment, the microspheres consisting of co-polymer having DL-lactide and glycolide in a molar ratio of 50:50 and 75:25 with an inherent viscosity ranging from 0.15 to 0.60 dL/g with either an ester or acid end group plus an antibiotic Vancomycin hydrochloride (VH) was formulated using the PN system. The loading capacity of the VH loaded microspheres was around 6% w/w with a mean diameter in the range of 10-50 micrometer. VH microspheres showed a steady release of VH over a period of 4-6 weeks.

### Embodiment B: Simultaneous Double Emulsion

In another embodiment of the presented technology, the microfluidic device was provided with the flow focusing junctions for double emulsion production. These two flow-focusing junctions aligned on the same axis and the areas of the inlets and outlets of the channels were designed to maximize the loading capacity of antibiotics in PLGA microparticles. The areas of the first feeding channels at the first flow focusing junction were the same for each channel. However, area of the outlet of the flow focusing junction was defined as 150% of the inlet channel areas at the beginning of the connecting channel and was increased to 200% before joining to the second flow focusing junction. Similarly, the areas of the other inlet channels that connected to the second flow focusing junction were doubled. Therefore, the inner water droplet of the double emulsion including the antibiotics was formed and coated with the PLGA layer and transferred into water phase, with a stabilizing agent such as PVA, to form the double emulsion. Vancomycin hydrochloride encapsulating PLGA microspheres were synthesized with up to 14% loading.

### EXAMPLES

### Example 1—Preparation of Solutions Used in Microparticle Fabrication

We prepared the solutions needed in the fabrication of microparticles, namely the organic phase, first and second water phases, parting solution, and the solution used in the collection container where the microparticles are collected.

The organic phase was a solution of PLGA in ethyl acetate. The first water phase was an aqueous solution of vancomycin hydrochloride. The second water phase, parting solution, and the solution in the collection container were the aqueous solution of PVA and NaCl. These solutions were used in the examples below with varying concentrations. In each example, the concentrations of the solutions are indicated in Table 1

TABLE 1
Details of the solutions used in the fabrication of PLGA microparticles as described in Examples 5 to 8.

| | | Solute | | Solvent | | |
|---|---|---|---|---|---|---|
| | | mass | | Volume | Solution | |
| Code | Solute | (mg) | Solvent | (ml) | Concentration | |
| S1.1 | Acid terminated PLGA (Resomer 503H) with | 300 | Ethyl acetate | 6 | 50 | mg/ml |
| | lactide:glycolide (L/G) ratio of 50:50 and | | | | | |
| | molecular weight of 24,000-38,000 | | | | | |
| S1.2 | Acid terminated PLGA (Resomer 503H) with L/G | 420 | Ethyl acetate | 6 | 70 | mg/ml |
| | ratio of 50:50 and molecular weight of 24,000- | | | | | |
| | 38,000 | | | | | |
| S2 | Vancomycin hydrochloride | 200 | Deionized water | 1 | 200 | mg/ml |
| S3.1 | Polyvinyl alcohol (PVA) with a molecular weight | 50 | 0.5N sodium | 100 | 0.05% | w/v |
| | of 13,000-23,000, hydrolyzed by 87-89% | | chloride in | | | |
| | | | deionized water | | | |
| S3.2 | PVA with a molecular weight of 13,000-23,000, | 1000 | 0.5N sodium | 100 | 1% | w/v |
| | hydrolyzed by 87-89% | | chloride in | | | |
| | | | deionized water | | | |

### Example 2. API Concentration in Solution Using a Calibration Curve

The concentration(s) of API(s) was determined to quantify the loading capacity and analyze the elution rate of API(s) from the microparticles. A calibration curve for each API solution was prepared to determine the concentration of the said API in that solution. Solutions were prepared with known concentrations in the range of 1-1000 μg/ml. High-performance liquid chromatography (HPLC) and/or ultraviolet (UV)-visible spectrophotometer plate reader were used to analyze the concentration of API solutions. Area under the curve (AUC) and spectrometric absorbance (ABS) were determined from the calibration curves.

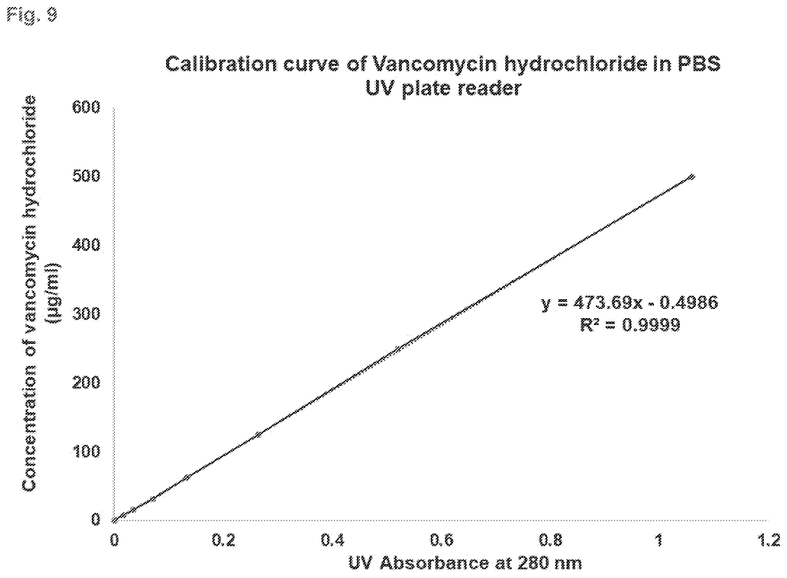

The calibration curve for vancomycin hydrochloride was prepared using a UV-Vis plate reader. A 1000 μg/ml stock solution of vancomycin hydrochloride in PBS. The stock solution was diluted with PBS to 500, 250, 125, 65.2, 31.25, 15.65, and 7.82 μg/ml. Each solution was analyzed to determine the UV-Vis absorbance (ABS) by placing 200 μL of each solution into a clear 96-well UV-Vis microplate for analysis at 280 nm, maximum absorbance wavelength for vancomycin hydrochloride, using UV Visible plate reader (BioTek Synergy H1. CA, USA). The linear regression between the calibration solutions and corresponding absorbances measured at 280 nm was used as the calibration curve (FIG. 8.).

### Example 3. Determination of Loading Capacity

The microparticles comprise the resorbable polymer and the API(s). Loading capacity (LC, (% w/w)) is the percent amount of drug loaded per unit mass of the microparticles. To analyze the loading capacity, the microparticles were mixed with an organic solvent, such as chloroform or dichloromethane, to solubilize the resorbable polymer, e.g., PLGA, and PBS to solubilize the API. The mixture was left to phase separate and/or centrifuged to separate the organic and PBS phases. The PBS phase comprising the solubilized API was collected and analyzed using for a UV-Vis plate reader, to determine the concentration of API in PBS.

For vancomycin hydrochloride loaded microparticles, the mass of lyophilized microparticles, M_{MP}, was determined by using a scale. The weighed particles were then solubilized in 2 mL of chloroform and known volume of PBS (V_{PBS}) mixture by vortexing for 2 minutes at 3000 rpm followed by stirring for 120 minutes. The stirring was to help dissolve microparticles and allow the API to be solubilized in PBS. After stirring, the mixture was left to phase separate for 15 minutes. The resulting aqueous supernatant comprising the previously encapsulated vancomycin hydrochloride was centrifuged at 15,000 rpm for 5 minutes. About 200 μL of supernatant was collected and analyzed with UV-Vis plate reader. Dilution was necessary if the supernatant was concentrated enough to saturate the API concentration readings on the UV-Vis plate reader. Dilution Factor (DF) was ratio of the volume of diluted sample to the volume of the supernatant collected after centrifuging. The supernatant or the diluted supernatant was then analyzed to determine the concentration of the API. Loading capacity was calculated using the following equations.

$$LC = \frac{{V}_{PBS} \times D F \times [ API ]}{{M}_{M P}} \times 100 \qquad ( 1 )$$

where [API] (mg/ml) is concentration of API in supernatant, M_{API} is mass of encapsulated API in mg, V_{PBS} is the volume of PBS (ml) used in organic solvent/PBS mixture used in dissolving the microparticles, DF is the supernatant dilution factor.

For example, the concentration of vancomycin hydrochloride was calculated with the UV-Vis absorbance of the samples at 280 nm wavelength (ABS), and slope of the calibration curve (FIG. 8)

$$[ API ] = A B S \times {m}_{cc} \qquad ( 2 )$$

Where _{ABS} is UV-Vis absorbance from plate reader, and m_{cc} is the slope of the calibration curve.

### Example 4. Elution Rate Quantification

The elution experiments were carried out at 37° C. with 100 rpm in release media. The said media were collected at regular intervals and analyzed to determine the mass of API released from a known amount of API loaded microparticles. The elution from API encapsulated PLGA microparticles was quantified using the LC-MS or UV-Vis plate reader method. The elution media were collected at different time intervals and analyzed with the LC-MS or UV-Vis plate reader to determine the concentration of API in the samples. The total mass of API released from the microparticles during each time interval was calculated as well as the cumulative percentage API elution, which is the ratio of cumulative API mass eluted as a function of elution time to the total amount of API loaded in the microparticles used in the elution experiment. Finally, elution rate is calculated as the ratio of the difference between the cumulative API mass measured at consecutive time intervals to time interval difference between the previous and current time points.

For example, the vancomycin hydrochloride encapsulated microparticles used for release were weighed and placed in a 5 ml centrifuge tube with a known volume of the release media. The release media was 0.01% w/v polyoxyethylene sorbitol ester (Tween 20) solubilized in phosphate buffer saline, PBS, (pH 7.4). The centrifuge tubes were placed on a temperature-controlled benchtop shaker at 100 rpm and 37° C. At varying time intervals in (ti), all the release media was collected from the tubes. If necessary, release media samples were diluted with a certain DF, and the samples were analyzed to determine the released API concentration, [API], (Eq 2). Subsequently, fresh release media was added to the tube to continue the elution experiments. The mass of vancomycin hydrochloride eluted from the microparticles, the cumulative mass of eluted vancomycin hydrochloride, percent cumulative vancomycin hydrochloride elution, and elution rate were determined by using the following equations:

$${M}_{{API}_{R}} = VR \times DF \times [ {API}_{R} ] \qquad ( 3 )$$

where M_{API}_{_{R}} is the mass of API eluted from microparticles in mg, VR is the volume of release media in ml, DF is the dilution factor, and [API_{R}] is concentration of the released API in mg/ml.

$${M}_{APIc} at {t}_{n} = {\sum }_{1}^{n} {M}_{{API}_{R}} at {t}_{i} \qquad ( 4 )$$

where M_{APIc} is the cumulative mass of API eluted from microparticles at a given time interval in mg, (ti) is the time interval in days of sample collection.

$$\% {M}_{APIc} at {t}_{n} = \frac{{\sum }_{1}^{n} {M}_{{API}_{R}} at {t}_{i}}{{M}_{API}} \times 100 \qquad ( 5 )$$

where % M_{APIc} is the ratio of the cumulative eluted API at a given time point to the total amount of API loaded into the microparticles in % mg/mg units.

Finally, the elution rate was determined by using Eq 6 Where ER (mg/day) at tn is the elution rate at a certain time interval in days.

$$ER at {t}_{n} = \frac{{M}_{APIc} at {t}_{n} - {M}_{APIc} at {t}_{n - 1}}{{t}_{n} - {t}_{n - 1}} \qquad ( 6 )$$

### Example 5. Formulation of Vancomycin Hydrochloride Loaded Poly (D, L-Lactide-Co-Glycolide) (PLGA) Microparticles with Two YJMDs

We performed two experiments, F1 and F2, to fabricate PLGA microparticles using two YJMDs assisted by syringe pumps. In these formulations, syringes were filled with o and w1 phases, syringe outlets were connected to the respective inlets of the first YJMD, and the syringes were placed in syringe pumps.

TABLE 2
Processing parameters used in the fabrication of microparticles.
The solutions used in the fabrication of F1 and F2 are detailed
in Table 1. PE is the primary emulsion. PE column shows the flow
rate of the primary emulsion as it was injected into the second
YJMD. Also shown are the loading capacities achieved with each trial.

| | Phases Used | | | Flowrates (ml/min) | | | | |
|---|---|---|---|---|---|---|---|---|
| | o | w1 | w2 | o | w1 | PE* | w2 | LC (% w/w) |
| F1 | S1.1 | S2 | S3.1 | 12.6 | 1.4 | 0.57 | 3.43 | 12.2 |
| F2 | S1.2 | S2 | S3.1 | 16.2 | 1.8 | 1.33 | 2.67 | 13.1 |

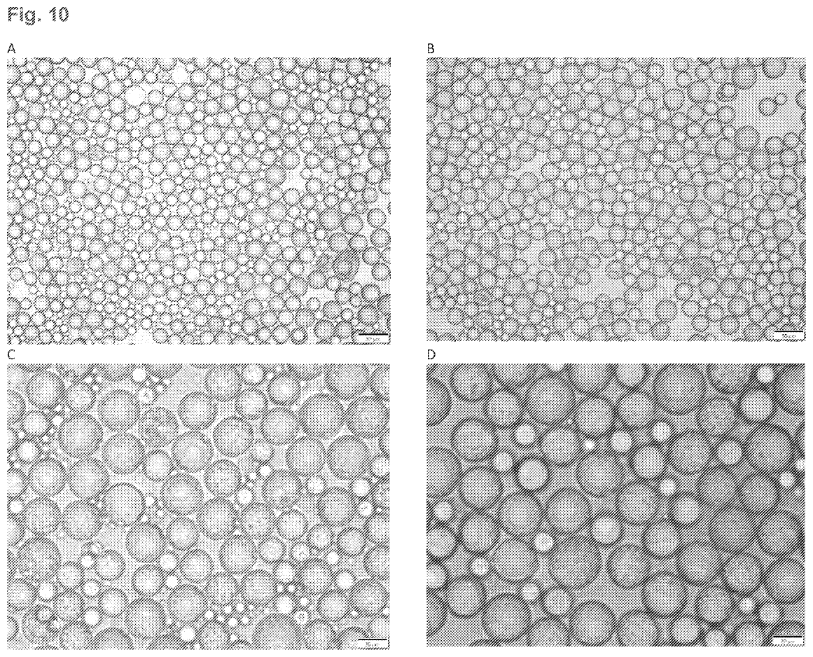

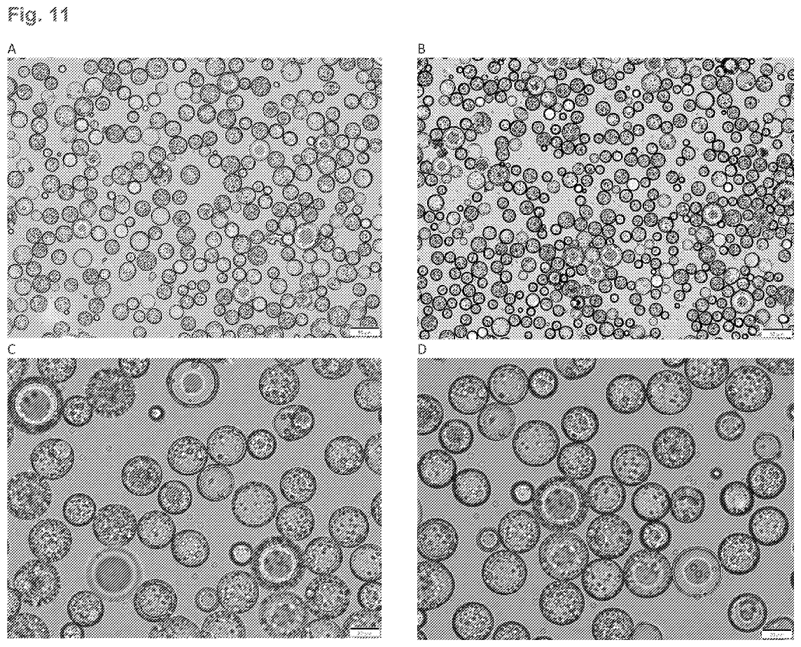

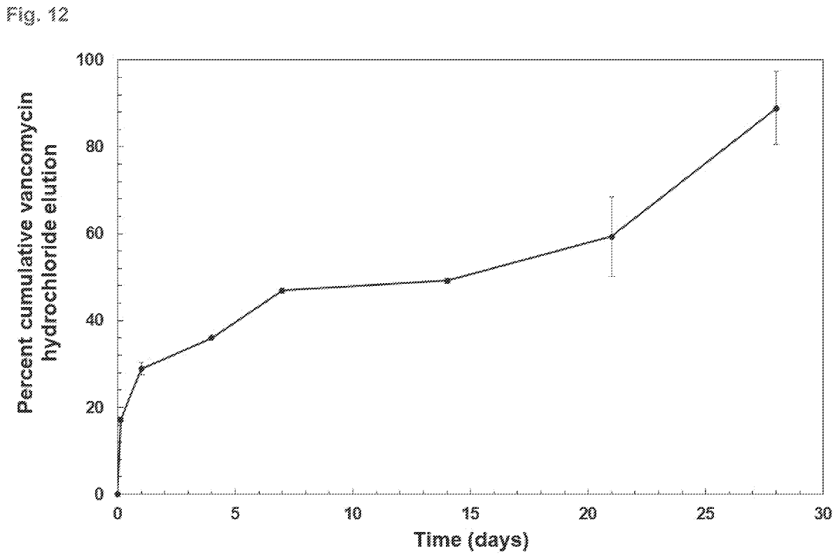

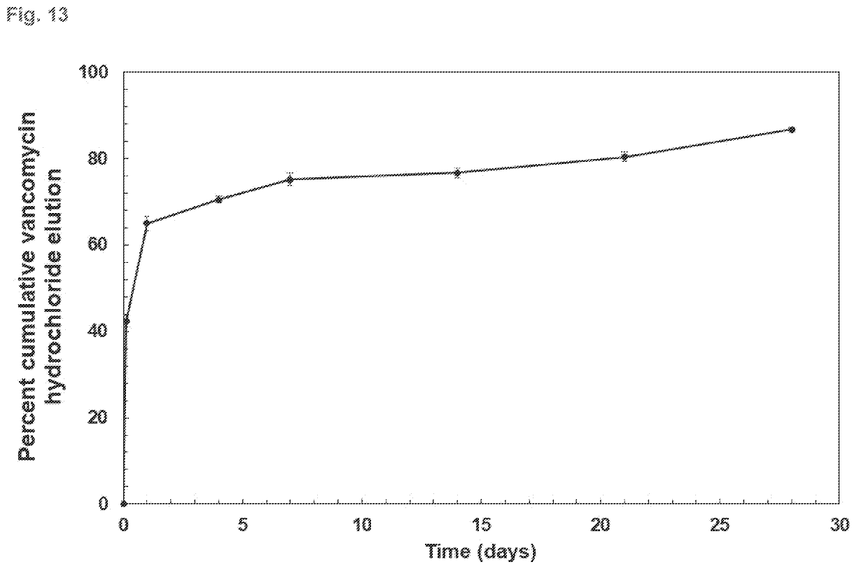

We controlled the flow rates of each solution with the syringe pumps to formulate the primary emulsion (w1/o). Solutions were pumped into the first YJMD at the corresponding flowrates shown in Table 2. We collected the primary emulsion from the first YJMD in a 50 ml plastic tube and transferred it to a syringe, which we connected to the primary emulsion inlet of the second YJMD. We connected another syringe comprising w2 phase to the w2 inlet of the second YJMD and mounted the syringes on the syringe pumps. We controlled flowrates of the primary emulsion and w2 phase as listed in Table 2. We collected the secondary emulsion (SE) in a 50 ml plastic tube, which contained 5-10 ml of w2 solution (Table 2). We pored the contents of the plastic tube into a beaker and placed it on a shaker for 12-16 h at 250 rpm at ambient temperature to allow evaporation of ethyl acetate and water. We placed the resulting dispersion in a centrifuge tube and centrifuged at 3000 rpm. We collected the microparticles in the form of a pellet. The pellet remained in the bottom of the tube and the supernatant was discarded. The pellet was then subjected to two cycles of washing and centrifugation. The washing cycles were done by adding 40 ml of deionized water into the tube and placing the tube on a vortex mixer for about two minutes at 3000 rpm. After each washing cycle, the tube was centrifuged at 2000 rpm and the supernatant was discarded to remove vancomycin hydrochloride that was not encapsulated and to remove any excess PVA. We added 5 ml of water into the tube comprising the microparticle pellet and subjected it to freezing at −80° C. for 12 hrs. The frozen pellet was then lyophilized using a freeze dryer for 48 h. Optical microscopy imaging confirmed the formation of microparticles with a spherical structure (FIG. 9, and FIG. 10.). We determined the loading capacity and elution rate using the methods described in Examples 2 and 3, respectively. The loading capacity of F1 and F2 were 12.2% and 13.1%, respectively (Table 2). The elution of vancomycin hydrochloride from these microparticles as a function of time is shown in FIG. 11. and FIG. 12.

### Example 6. Formulation of Vancomycin Hydrochloride Loaded Poly (D, L-Lactide-Co-Glycolide) (PLGA) Microparticles with DYJMD

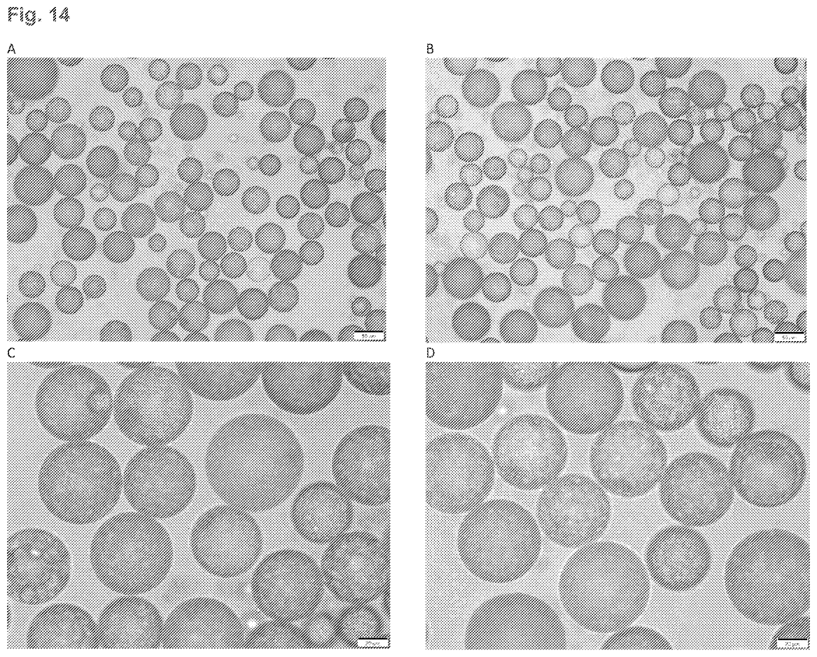

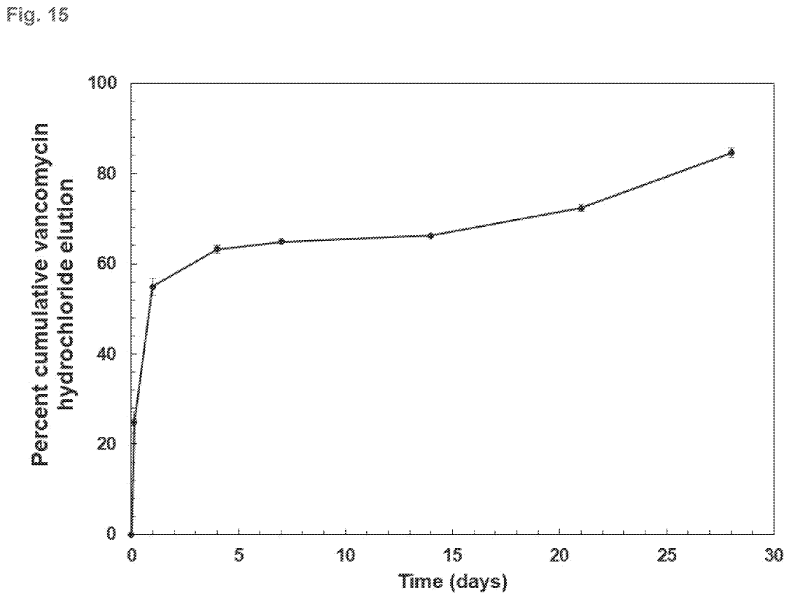

We fabricated PLGA microparticles (F3) using one DYJMD assisted by syringe pumps. Syringes were filled with o (Table 1 S1.1), w1 (Table 1 S2), and w2 (Table 1 S3.2) phases, syringe outlets were connected to the respective inlets of the DYJMD, and the syringes were placed in syringe pumps. We controlled the flowrates of each solution at 12.6 ml/min, 1.4 ml/min, and 28 ml/min for the o, w1, and w2 phases, respectively, with the syringe, and pumps to formulate the secondary emulsion. We collected the secondary emulsion (SE) from the outlet in a 50 ml plastic tube, which contained 5-10 ml of w2 solution. The contents of the plastic tube were poured into a beaker and placed on a shaker for 12-16 h at 250 rpm at ambient temperature to evaporate ethyl acetate and water. The resulting dispersion was placed in a centrifuge tube. The microparticles in this dispersion were then collected in the form of a pellet by centrifugation at 3000 rpm. The pellet remained in the bottom of the tube and the supernatant was discarded. The pellet was then subjected to two cycles of washing and centrifugation. The washing cycles were done by adding 40 ml of deionized water into the tube and placing the tube on a vortex mixer for about two minutes at 3000 rpm. After each washing cycle, the tube was centrifuged at 2000 rpm and the supernatant was discarded to remove vancomycin hydrochloride that was not encapsulated and to remove any excess PVA. We added 5 ml of water into the tube comprising the microparticle pellet and subjected it to freezing at −80° C. for 12 hrs. The frozen pellet was then lyophilized using a freeze dryer for 48 h. Optical microscopy imaging confirmed the formation of microparticles with a spherical structure (FIG. 13.). We determined the loading capacity and elution rate using the methods described in Examples 2 and 3, respectively. The loading capacity of vancomycin hydrochloride-loaded microparticles was 20.7% w/w. The elution of vancomycin hydrochloride from these microparticles as a function of time is shown in FIG. 14.

### Example 7. Formulation of Vancomycin Hydrochloride Loaded Poly (D, L-Lactide-Co-Glycolide) (PLGA) Microparticles without Parting the Channel

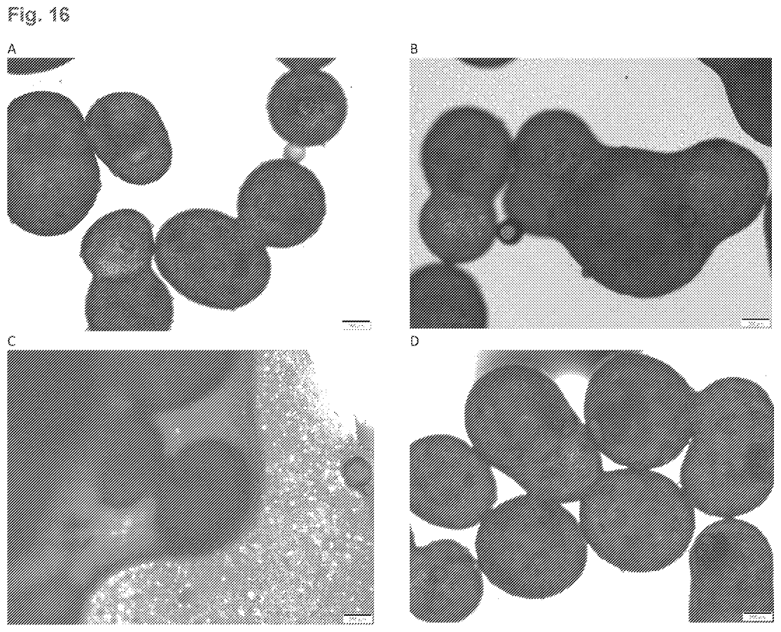

We fabricated PLGA microparticles (F4) using one DFFMD assisted by syringe pumps. Syringes were filled with o (Table 1 S1.1), w1 (Table 1 S2), and w2 (Table 1 S3.1) phases, syringe outlets were connected to the respective inlets of the DFFMD, and the syringes were placed in the syringe pumps. We controlled the flowrates of each solution at 5.4 ml/min, 0.6 ml/min, and 8 ml/min for the o, w1, and w2 phases, respectively, with the syringe, and pumps connected to the inlets. The outlet of the DFFMD was connected to a beaker and the beaker was placed on a shaker for 12-16 h at 250 rpm at ambient temperature to evaporate ethyl acetate and water. The resulting dispersion was placed in a centrifuge tube. The microparticles in this dispersion were then collected in the form of a pellet by centrifugation at 3000 rpm. The pellet remained in the bottom of the tube after centrifugation and the supernatant was discarded. The pellet was then subjected to two cycles of washing and centrifugation. The washing cycles were by adding 40 ml of deionized water into the tube and placing the tube on a vortex mixer for about two minutes at 3000 rpm. After each washing cycle, the tube was centrifuged at 2000 rpm and the supernatant was discarded to remove vancomycin hydrochloride that was not encapsulated and to remove any excess PVA. We added 5 ml of water into the tube comprising the microparticle pellet and subjected it to freezing at −80° C. for 12 hrs. The frozen pellet was then lyophilized using a freeze dryer for 48 h. Optical microscopy confirmed the formation of microparticles with spherical structures (FIG. 15.) and aggregated microparticles forming fiber-like structures.

### Example 8. Formulation of Vancomycin Hydrochloride Loaded Poly (D, L-Lactide-Co-Glycolide) (PLGA) Microparticles with Parting Channel

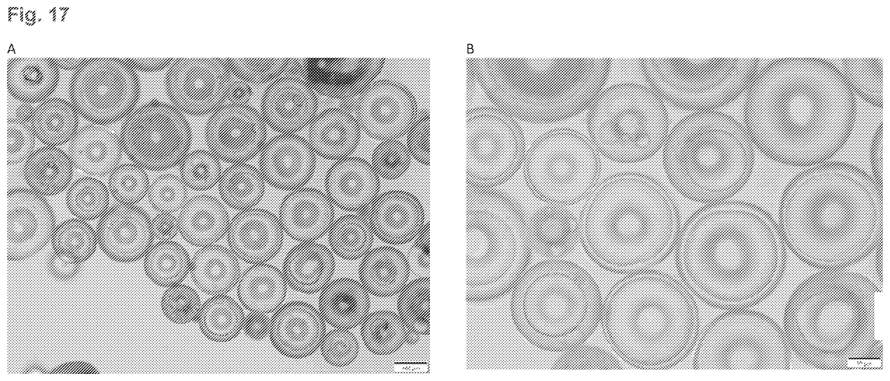

We fabricated PLGA microparticles (F5) using one DFFMD assisted by syringe pumps. Syringes were filled with o (Table 1 S1.1), w1 (Table 1 S2), and w2 (Table 1 S3.1) phases, syringe outlets were connected to the respective inlets of the DFFMD, and the syringes were placed in syringe pumps. We controlled the flowrates of each solution at 5.4 ml/min, 0.6 ml/min, and 8 ml/min for the o, w1, and w2 phases, respectively, with the syringe, and pumps connected to the inlets. The outlet of the DFFMD (FIG. 5) was connected to a parting channel that fed the parting solution to the microfluidic device at a rate of 8 ml/min with an additional syringe pump. The fluid mixture, including the emulsion and parting solution, was collected in a beaker. The beaker was placed on a shaker for 12-16 h at 250 rpm at ambient temperature to evaporate ethyl acetate and water. The resulting dispersion was placed in a centrifuge tube. The microparticles in this dispersion were then collected in the form of a pellet by centrifugation at 3000 rpm. The centrifuged pellet remained in the bottom of the tube and the supernatant was discarded. The pellet was then subjected to two cycles of washing and centrifugation. The washing cycles were done by adding 40 ml of deionized water into the tube and placing the tube on a vortex mixer for about two minutes at 3000 rpm. After each washing cycle, the tube was centrifuged at 2000 rpm and the supernatant was discarded to remove vancomycin hydrochloride that was not encapsulated and to remove any excess PVA. We added 5 ml of water into the tube comprising the microparticle pellet and subjected it to freezing at −80° C. for 12 hrs. The frozen pellet was then lyophilized using a freeze dryer for 48 h. Optical microscopy confirmed the formation of microparticles with spherical structures (FIG. 16.). This method prevented most of the aggregation and resulted in fewer fiber-like structures.

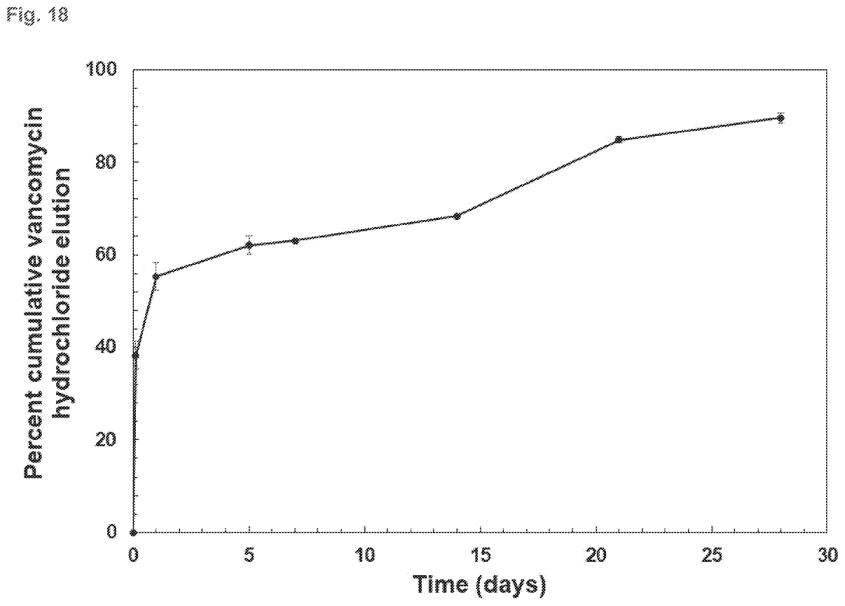

We determined the loading capacity and elution rate using the methods described in Examples 2 and 3, respectively. The loading capacity of the vancomycin hydrochloride loaded microparticles was 13.5% w/w. The elution of vancomycin hydrochloride from these microparticles as a function of time is shown in FIG. 17.

### Example 9. Formulation of Rifampicin Loaded Poly (D, L-Lactide-Co-Glycolide) (PLGA) Microparticles with One YJMD

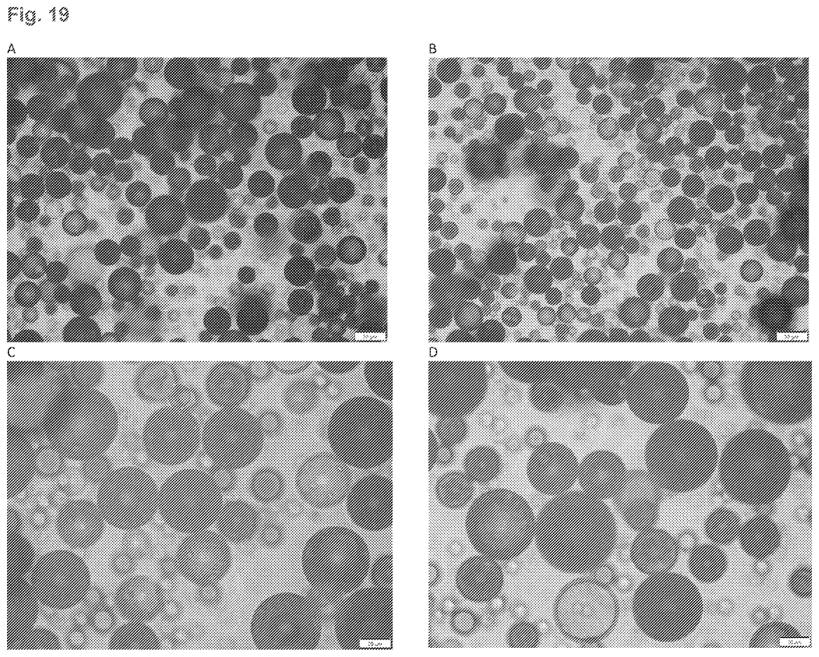

We fabricated PLGA microparticles (F6) using a single YJMD assisted by syringe pumps. The organic phase (o) was prepared by solubilizing 280 mg of PLGA and 70 mg of rifampicin in 2 ml of ethyl acetate. The aqueous phase (w) was prepared as mentioned in Table 1 S3.1. In this formulation, syringes were filled with o and w phases, syringe outlets were connected to the respective inlets of YJMD, and the syringes were placed in syringe pumps. We controlled the flow rates of each solution (that is o and w phases) with the syringe pumps to formulate the single emulsion (o/w). Solutions were pumped at 4 ml/min. We collected the single emulsion in a 50 ml plastic tube, which contained 5-10 ml of w solution (Table 1 S3.1). The contents of the plastic tube were poured into a beaker and placed on a shaker for 12-16 h at 250 rpm at ambient temperature to allow evaporation of ethyl acetate and water. The resulting dispersion was placed in a centrifuge tube. The microparticles in this dispersion were then collected in the form of a pellet by centrifugation at 3000 rpm. The pellet remained in the bottom of the tube and the supernatant was discarded. The pellet was then subjected to two cycles of washing and centrifugation. The washing cycles were done by adding 40 ml of deionized water into the tube and placing the tube on a vortex mixer for about two minutes at 3000 rpm. After each washing cycle, the tube was centrifuged at 2000 rpm and the supernatant was discarded to remove rifampicin that was not encapsulated and to remove any excess PVA. We added 5 ml of water into the tube comprising the microparticle pellet and subjected it to freezing at −80° C. for 12 hrs. The frozen pellet was then lyophilized using a freeze dryer for 48 h. Transmission optical microscopy imaging confirmed the formation of microparticles with a spherical structure as shown in FIG. 18.

It will be appreciated by those skilled in the art that while the disclosed subject matter is described above in connection with particular embodiments and examples, the invention is not necessarily so limited, and that numerous other embodiments, examples, uses, modifications and departures from the embodiments, examples and uses are intended to be encompassed by the claims attached hereto. Each article cited herein is incorporated by reference in its entirety.

While the invention has been described with reference to certain example embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of devices would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents. Various features and advantages of the invention are set forth in the following claims.

We claim:

1. A method of treating a patient comprises administering to said patient a therapeutically effective amount of an injectable formulation comprising microparticles, the microparticles comprising at least one active pharmaceutical ingredient and a poly(lactic-co-glycolic) acid copolymer (PLGA) matrix, said microparticles manufactured by microfluidic devices.

2. Microparticles of claim 1 manufactured by microfluidic devices using a single emulsion technique.

3. Microparticles of claim 1 manufactured by microfluidic devices using the double emulsion method.

4. Microparticles of claim 1 wherein the microparticles comprise at least 5% by weight of one or more infection management active pharmaceutical ingredients.

5. A microparticle formed of a PLGA matrix of claim 1 wherein the PLGA matrix comprises a lactic acid to glycolic acid molar ratio of 50/50 or 75/25 or of from 50/50 or 75/25.

6. The method of claim 1 wherein the active pharmaceutical ingredient is vancomycin hydrochloride, gentamicin sulfate, rifampicin, or tobramycin, or a combination thereof.

7. The method of claim 1, wherein the injectable formulation further comprises a dilution fluid.

8. The method of claim 1, wherein the injectable formulation is administered as one or more intra-articular injections.

9. The method of claim 1, wherein the active pharmaceutical ingredient is released for between at least 7 days and 90 days following administration to a patient.

10. The method of claim 1, wherein the loading capacity of the active pharmaceutical ingredient in the microparticles is above 5%, from 5% to 90%, or from 10% to 80%.

11. The method of claim 1, wherein the microparticles further comprise a polyethylene glycol (PEG).

12. The method of claim 1, wherein the patient is human.

13. The method of claim 1, wherein the patient has a postsurgical infection or is undergoing surgery or the administering is before the patient undergoes surgery.

14. A method of making microparticles comprising at least one infection management active pharmaceutical ingredient the method comprising the steps of
delivering an organic phase fluid to a first channel on a microfluidic device, the organic phase comprising a solvent and a polymer material;
delivering a first aqueous phase fluid to a second channel on the microfluidic device, the first aqueous phase comprising water;
wherein an active pharmaceutical ingredient is provided in either or both of the organic phase fluid and the first aqueous phase fluid, and the first channel and the second channel on the microfluidic device intersect at a first junction so that the organic phase fluid and the first aqueous phase fluid come in contact to form an emulsion, enter a third channel and form microparticles in the fluid; and
removing the fluid comprising microparticles from the microfluidic device.

15. The method of claim 14 wherein the polymer material is a poly(lactic-co-glycolic) acid copolymer (PLGA) and the solvent is an organic solvent.

16. The method of claim 14 wherein the active pharmaceutical ingredient is vancomycin hydrochloride, gentamicin sulfate, rifampicin, or tobramycin, or a combination thereof.

17. (canceled)

18. (canceled)

19. (canceled)

20. (canceled)

21. (canceled)

22. The method of claim 14, further comprising delivering a second aqueous phase fluid to a fourth channel, the fourth channel having a second junction, the second junction intersecting the third channel.

23. (canceled)

24. The method of claim 14, further comprising delivering the fluid comprising microparticles into a first channel on a second microfluidic device and delivering a second aqueous phase fluid to a second channel on the second microfluidic device.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A microparticle produced by the method of claim 14.

37. A microfluidic device for producing emulsions comprising:
at least one microchannel connected to a flow source, the microchannel having a completely or partially void structure to form a cavity, the cavity allowing fluids, dispersions and/or emulsions to pass through the cavity, wherein at least one of the width, length or height of the channel is a maximum 1000 micrometers,
at least two fluid inlets connected to the microchannel and allowing fluid to enter the microchannel,
at least one fluid outlet connected to the channel and enables the fluid to exit from the microchannel,
at least two flow focusing junctions located between the fluid inlet and the fluid outlet in the channel, which changes the flow properties of the fluid in the channel to form emulsions in the channel.

38. (canceled)

39. (canceled)

40. (canceled)

41. (canceled)

42. (canceled)
